(19) United States
(12) Patent Application Publication
(10) Pub. No.: US 2004/0023361 A1
(43) Pub. Date: Feb. 5, 2004

(54) Lactic acid bacteria producing polysaccharide similar to those in human milk and corresponding gene

(75) Inventors: Walter Gaier, Chailly-Montreux (CH); David Pridmore, Lausanne (CH); Francesca Stingele, St-Prex (CH); Jean-Richard Neeser, Savigny (CH); Patrice Desachy, Porsel (CH); Bruno Pot, Sint-Michiels Brugge (BE)

Correspondence Address: WINSTON & STRAWN, PATENT DEPARTMENT, 1400 L STREET, N.W., WASHINGTON, DC, 20005-3502, US

(73) Assignee: NESTEC S.A.

(21) Appl. No.: 10/461,990

(22) Filed: Jun. 16, 2003

Related U.S. Application Data
(63) Continuation-in-part of application No. 09/548,606, filed on Apr. 13, 2000, now Pat. No. 6,579,711.
Application No. 09/548,606 is a continuation of application No. PCT/EP98/06636, filed on Oct. 9, 1998.

(30) Foreign Application Priority Data
Oct. 17, 1997 (EP) 97203245.2

(51) Int. Cl. A61K 31/715; C08B 37/00; C12N 1/20

(52) U.S. Cl. 435/252.9; 536/55.1; 514/54; 536/53

(57) ABSTRACT

A lactic acid bacterium having a 16S ribosomal RNA characteristic of the genus Streptococcus, cocci morphology, a growth optimum in the range of about 28° C. to about 45° C., having the ability to ferment D-galactose, D-glucose, D-fructose, D-mannose, and N-acetyl (D)-glucosamine, salicin, cellobiose, maltose, lactose, sucrose and raffinose, and imparting a viscosity of greater than 100 mPa.s at a shear rate of about 293 s⁻¹. The strain often produces an exopolysaccharide comprising a chain of glucose, galactose and N-acetylglucosamine in a proportion of 3:2:1 respectively. The new strain is identified as Streptococcus macedonicus. Other characteristics include a total protein profile obtained after culture in an MRS medium for 24 h at 28° C., extraction of the total proteins and migration of the proteins on an SDS-PAGE electrophoresis gel, exhibits a degree of Pearson correlation of at least 78 with respect to bacterium CNCM I-1920 or I-1926. The strain and its secreted polysaccharides can be used in preparing dietary compositions. The present invention further relates to a new exopolysaccharide synthesis operon and the genes thereof isolated from the new species and to transformed cells having inserted nucleotides that encode proteins of the EPS operon or at least one gene thereof.

### CROSS-REFERENCE TO RELATED APPLICATIONS

This application is a continuation-in-part of U.S. patent application Ser. No. 09/548,606, filed Apr. 13, 2000, which is a continuation of the U.S. national phase of International Application No. PCT/EP98/06636, filed Oct. 9, 1998, the content of both of which is expressly incorporated herein by reference thereto and claim priority to Swiss Patent Application No. 97203245.2 filed Oct. 17, 1997.

### FIELD OF THE INVENTION

The present invention relates to new species of lactic acid bacteria belonging to the genus Streptococcus, identified herein as Streptococcus macedonicus and its use in the production of food compositions. The present invention further relates to a new exopolysaccharide synthesis operon isolated from the new species Streptococcus macedonicus and transformed microorganisms containing the operon or genes thereof.

### BACKGROUND OF THE INVENTION

The identification of lactic acid bacteria is essential in the dairy industry, and consists of differentiating distinctive morphological, physiological and/or genetic characteristics between several species.

The distinctive physiological characteristics for a given species of lactic acid bacteria may be determined by various tests including, for example, analyzing their capacity to ferment various sugars and the migration profile of total proteins on an SDS-PAGE type electrophoresis gel (Pot et al., Taxonomy of lactic acid bacteria, in Bacteriocins of lactic acid bacteria, Microbiology, Genetics and Applications, L. De Vuyst and E. J. Vandamme ed., Blackie Academic & Professional, London, 1994).

The migration profile of the total proteins of a given species, determined by SDS-PAGE gel electrophoresis, when compared, with the aid of a densitometer, with other profiles obtained from other species, makes it possible to determine the taxonomic relationships between the species. Numerical analysis of the various profiles, for example, with the GelCompar® software, makes it possible to establish the degree of correlation between the species which is a function of various parameters, in particular of the algorithms used (GelCompar, version 4.0, Applied Maths, Kortrijk, Belgium; algorithms: “Pearson Product Moment Correlation Coefficient, Unweighted Pair Group Method Using Average Linkage”).

To date, comparative analysis of the total protein profile by SDS-PAGE gel electrophoresis has been thoroughly tested as an effective means for distinguishing between homogeneous and distinct groups of species of lactic acid bacteria (Pot et al., Chemical Methods in Prokaryotic Systematics, Chapter 14, M. Goodfellow, A. G. O'Donnell, Ed., John Wiley & Sons Ltd, 1994).

With this SDS-PAGE method, the preceding experiments have thus shown that when a degree of Pearson correlation of more than 78 (on a scale of 100) is obtained between two strains of lactic acid bacteria, it is justifiably possible to deduce therefrom that they belong to the same species (Kersters et al., Classification and Identification methods for lactic bacteria with emphasis on protein gel electrophoresis, in Acid Lactic Bacteria, Actes du Colloque Lactic '91, 33-40, Adria Normandie, France, 1992; Pot et al., The potential role of a culture collection for identification and maintenance of lactic acid bacteria, Chapter 15, pp. 81-87, in: The Lactic Acid Bacteria, E. L. Foo, H. G. Griffin, R. Mollby and C. G. Heden, Proceedings of the first lactic computer conference, Horizon Scientific Press, Norfolk).

By way of example, it was recently possible to divide the group of acidophilic lactic acid bacteria into 6 distinct species by means of this technique (Pot et al., J. General Microb., 139, 513-517, 1993). Likewise, this technique was recently used to establish, in combination with other techniques, the existence of several new species of Streptococcus, such as Streptococcus dysgalactiae subsp. equisimilis, Streptococcus hyo lis sp. nov. and Streptococcus thoraltensis sp. nov (Vandamme et al., Int. J. Syst. Bacteriol., 46, 774-781, 1996; Devriese et al., Int. J. Syst. Bacteriol., 1997, In press).

The identification of new species of lactic acid bacteria cannot however be reduced to a purely morphological and/or physiological analysis of the bacteria. To date, the “Deutsche Sammlung Von Mikroorganismen und Zellkulturen GmbH” (DSM, Braunschweig, Germany) has officially recorded about 48 different species belonging to the genus Streptococcus (see the list below). All these species possess a 16S ribosomal RNA that is typical of the genus Streptococcus, and may be divided into distinct and homogeneous groups by means of the SDS-PAGE technique mentioned above.

The present invention relates to the identification, by means of the techniques presented above, of a new species of lactic acid bacterium belonging to the genus Streptococcus, and to its use in the dairy industry in general.

As used herein, “biologically pure culture” means a culture free of deleterious viable contaminating microorganisms.

### SUMMARY OF THE INVENTION

The present invention relates to a new species of lactic acid bacteria belonging to the genus Streptococcus, identified herein as Streptococcus nacedonicus, and its use in the production of food compositions.

Streptococcus macedonicus has a 16S ribosomal RNA characteristic of the genus Streptococcus. Preferably the 16S ribosomal RNA characteristic of the new species Streptococcus macedonicus comprises a nucleic acid that is SEQ ID NO:1 or a homologue of SEQ ID NO:1 having 1-8 nucleotide substitutions, deletions, or additions, or more preferably only 1-4, and most preferably only 1-2. Other 16S rRNA characteristic of Streptococcus can be found at the GenBank database, for example under the accession numbers AF429762-AF429766.

The new species has cocci morphology and a growth optimum in the range of about 28° C. to about 45° C., and generally has the ability to ferment D-galactose, D-glucose, D-fructose, D-mannose, and N-acetyl(D)-glucosamine, salicin, cellobiose, maltose, lactose, sucrose and raffinose, and imparts a viscosity of greater than 100 mPa.s at a shear rate of about 293 s⁻¹ when used to ferment semi-skimmed milk at 38° C. at up to a pH 5.2.

Preferably the strain of Streptococcus macedonicus has 16S ribosomal RNA has a nucleotide sequence that is SEQ ID NO:1. Furthermore, strains of Streptococcus macedonicus advantageously produce an exopolysaccharide having a chain of glucose, galactose and N-acetylglucosamine in a proportion of 3:2:1 respectively. These exopolysaccharides are useful in the preparation of food compositions, especially diary products. The polysaccharides can also be hydrolyzed and used in hypoallergenic compositions that are desired for use in infant products and are similar to polysaccharides found in human milk.

Strains of Streptococcus macedonicus typically have a total protein profile obtained after culture of the bacterium in an MRS medium for 24 h at 28° C., extraction of the total proteins and migration of the proteins on an SDS-PAGE electrophoresis gel, and exhibit a degree of Pearson correlation of at least 78 with respect to the profile obtained under identical conditions with the strain of lactic acid bacterium CNCM I-1920 or I-1926.

The present invention further relates to a new exopolysaccharide synthesis operon isolated from the new species Streptococcus macedonicus and identified as SEQ ID NO:4 and to the specific genes and peptides produced and identified as SEQ ID NOS:5, 6, 8-13, 15, 18-36.

In one embodiment of the invention, a biologically pure culture of a lactic acid bacteria strain has a nucleotide sequence which encodes polypeptides identified by SEQ ID NOS: 18, 20, 22-24, 27, 28, 32, and 34 (SM-epsA, C, E-G, J, K, O, and Q), wherein the strain produces an exopolysaccharide comprising a chain of glucose, galactose and N-acetylglucosamine in a proportion of 3:2:1 respectively

Preferably the strain also comprises a nucleotide sequence encoding polypeptides identified by SEQ ID NOS:21, 25-26, and 33 (SM-epsD, H-I, and P) and still more preferably the strain also comprises a nucleotide sequence that encodes the polypeptides identified by SEQ ID NOS:19 and 29-31 (SM-epsB and L-N). In one embodiment the strain comprises SEQ ID NO:4.

The present invention further encompasses the isolated EPS operon (SEQ ID NO:4), genes thereof, and nucleotide sequences that encode the peptides of the EPS operon and preferably those identified by SEQ ID NO:25 (SM-epsH), SEQ ID NO:26 (SM-epsI), or SEQ ID NO:35 (SM-epsR).

Another aspect of the invention is use of the isolated nucleotides, or nucleotide sequences that encode peptides of the EPS operon, to transform a cell. Preferably the transformed cell is a microorganism that contains the Streptococcus macedonicus EPS operon or at least one of the genes of the operon and produces an exopolysaccharide comprising a chain of glucose, galactose and N-acetylglucosamine in a proportion of 3:2:1 respectively when cultured in milk.

In a further embodiment, the invention relates to any lactic acid bacterium, whose 16S ribosomal RNA is characteristic of the genus Streptococcus; and whose total protein profile, obtained after migration of the total proteins on an SDS-PAGE electrophoresis gel, is characteristic of that of the strain of lactic acid bacterium CNCM I-1920, but distinct from those of the recognized species belonging to the genus Streptococcus, namely S. acidominimus, S. agalactiae, S. alactolyticus, S. anginosus, S. bovis, S. canis, S. caprinus, S. constellatus, S. cricetus, S. cristatus, S. difficile, S. downei, S. dysgalactiae ssp. dysgalactiae, S. dysgalactiae ssp. equismilis, S. equi, S. equi ssp. equi, S. equi ssp. zooepidemicus, S. equinus, S. ferus, S. gallolyticus, S. gordonii, S. hyointestinalis, S. hyo lis, S. iniae, S. intermedius, S. intestinalis, S. macacae, S. mitis, S. mutans, S. oralis, S. parasanguinis, S. parauberis, S. phocae, S. pleomorphus, S. pneumoniae, S. porcinus, S. pyogenes, S. ratti, S. salivarius, S. sanguinis, S. shiloi, S. sobrinus, S. suis, S. thermophilus, S. thoraltensis, S. uberis, S. vestibularis, S. viridans.

A further aspect of the invention is use of a strain of lactic acid bacterium according to the invention for the preparation of a dietary composition, in particular an acidified milk or a fromage frais, for example.

The invention also relates to the use of a polysaccharide, capable of being secreted by a lactic acid bacterium according to the invention, which consists of a chain of glucose, galactose and N-acetylglucosamine in a respective proportion of 3:2:1, for the preparation of a dietary or pharmaceutical composition.

The subject of the invention yet further encompasses a dietary or pharmaceutical composition comprising a strain of lactic acid bacterium according to the invention.

Finally, the subject of the invention is also a dietary or pharmaceutical composition comprising a polysaccharide consisting of a chain of glucose, galactose and N-acetylglucosamine in a respective proportion of 3:2:1.

### DESCRIPTION OF THE DRAWINGS

FIG. 1 is a photographic depiction of the migration profiles of the total proteins of several strains of the new species, on an SDS-PAGE electrophoresis gel, in comparison with those obtained with Streptococcus thermophilus strains. The degree of filiation of the strains is indicated with the aid of the Pearson correlation scale and by means of a tree opposite the protein profiles (the degrees of Pearson correlation of 55 to 100 are represented).

FIG. 2 is a depiction the graditherm for the strain CNCM I-1920.

FIG. 3 is an alignment of the S. macedonicus I-1923 epsA PCR amplification product (SEQ ID NO:15) (upper strand) to the S. thermophilus Sfi6 epsA sequence (SEQ ID NO:16) (lower strand). Note the 10 base-pair deletion at approximately position 830 in the I-1923 sequence.

FIG. 4 is a diagram of an inverted PCR template and primer pair design strategy.

FIG. 5 shows the strategy for the confirmation of the sequenced DNA used.

FIG. 6 is a schematic map of the S. macedonicus exopolysaccharide synthesis operon.

FIG. 7 shows the ribosome-binding sites for the predicted S. macedonicus eps synthesis genes. The sequences were aligned backwards from the translation initiation codon for each gene and the predicted ribosome-binding sites are underlined.

FIG. 8 shows the DNA sequence and predicted translation products of the S. macedonicus strain I-1923 exopolysaccharide synthesis operon (SEQ ID NO:4). Probable translation initiation and termination codons are boxed, while predicted ribosome-binding sites are underlined.

FIG. 9 is an alignment comparison of the S. pneumoniae serotype 33f cap33fM protein (SEQ ID NO:17) (upper sequence) to the predicted S. macedonicus SM-epsP protein (SEQ ID NO:31) (lower sequence). Internal translation termination sites are indicated with a large X in red.

FIG. 10 is a comparison of the I-1923 eps operon DNA sequences surrounding the IS element to the SC147 eps operon without IS element.

FIG. 11 is a schematic for the synthesis of the repeating oligosaccharide unit in S. macedonidus I-1923.

### DETAILED DESCRIPTION OF THE INVENTION

The newly discovered species of the invention is of the genus Streptococcus, referred to herein as Streptococcus macedonicus. Identification of Streptococcus macedonicus is preferably demonstrated by comparing the nucleotide sequence of the 16S ribosomal RNA of the bacteria of the invention, or of their genomic DNA that encodes for the 16S ribosomal RNA, with those of other genera and species of lactic acid bacteria known to date. More particularly, it is possible to use the method disclosed in Example 1 below, or alternatively other methods known to a person skilled in the art, for example, as set forth in Schleifer et al., System. Appl. Microb., 18, 461-467, 1995; Ludwig et al., System. Appl. Microb., 15, 487-501, 1992. The nucleotide sequence SEQ ID NO:1 presented in the sequence listing below is an example of a 16S ribosomal RNA sequence that is characteristic of the new species of lactic acid bacteria, and exhibits striking similarities with the 16S ribosomal RNA sequences found in the species of Streptococcus recognized to date. Preferably the 16S ribosomal RNA characteristic of the new species Streptococcus macedonicus comprises a nucleic acid that is SEQ ID NO:1 or a homologue of SEQ ID NO:1 having 1-8 nucleotide substitutions, deletions, or additions, or more preferably only 1-4, and most preferably only 1-2. Other 16S rRNA characteristic of Streptococcus can be found at the GenBank database, for example under the accession numbers AF429762-AF429766.

The new species according to the invention, which constitutes a distinct and homogeneous new group, can also be differentiated from the other known species belonging to the,genus Streptococcus by means of the technique for identification of the total proteins by SDS-PAGE gel electrophoresis, described above.

In particular, this new species may give a total protein profile, obtained after culture of the bacterium in an MRS medium for 24 h at 28° C., extraction of the total proteins and migration of the proteins on an SDS-PAGE electrophoresis gel, which exhibits a degree of Pearson correlation of at least 78 (on a scale of 100) with the profile obtained under identical conditions with the strain of lactic acid bacterium CNCM I-1920 or I-1926.

More particularly, this technique consists of (1) isolating all the proteins (=total proteins) of a culture of lactic acid bacterium cultured under defined conditions, (2) separating the proteins by electrophoresis on an SDS-PAGE gel, (3) analyzing the arrangement of the different protein fractions separated with the aid of a densitometer which measures the intensity and the location of each band, (4) and comparing the protein profile thus obtained with those of several other species of Streptococcus which have been obtained, in parallel or beforehand, under exactly the same operating conditions.

The techniques for preparing a total protein profile as described above, as well as the numerical analysis of such profiles, are well known to a person skilled in the art. However, the results are only reliable insofar as each stage of the process is sufficiently standardized. Faced with this requirement, standardized procedures are regularly made available to the public by their authors such as that of Pot et al., as presented during a “workshop” organized by the European Union, at the University of Ghent, in Belgium, on 12 to 16 September 1994 (Fingerprinting techniques for classification and identification of bacteria, SDS-PAGE of whole cell protein).

The software used in the technique for analyzing the SDS-PAGE electrophoresis gel is of crucial importance since the degree of correlation between the species depends on the parameters and algorithms used by this software. Without going into the theoretical details, quantitative comparison of bands measured by a densitometer and normalized by a computer is preferably made with the Pearson correlation coefficient. The similarity matrix thus obtained may be organized with the aid of the UPGMA (unweighted pair group method using average linkage) algorithm that not only makes it possible to group together the most similar profiles, but also to construct dendograms (see K. Kerster-s, Numerical methods in the classification and identification of bacteria by electrophoresis, in Computer-assisted Bacterial Systematics, 337-368, M. Goodfellow, A. G. O'Donnell Ed., John Wiley and Sons Ltd, 1985).

Preferably, the strains of the new species exhibit a total protein profile having a degree of Pearson correlation of at least 85 with respect to one of the strains of bacteria of the new species. For the biotypes mentioned below, this degree of Pearson correlation can even exceed 90, for example.

By means of the SDS-PAGE electrophoresis gel technique for identification, the new species according to the invention that belong to the genus Streptococcus may be distinguished from all the species of Streptococcus recognized to date, namely S. acidominimus, S. agalactiae, S. alactolyticus, S. aoginosus, S. bovis, S. canis, S. caprinus, S. constellatus, S. cricetus, S. cristatus, S. difficile, S. downei, S. dysgalactiae ssp. dysgalactiae, S. dysgalactiae ssp. equisimilis, S. equi, S. equi ssp. equi, S. equi ssp. zooepidemicus, S. equinus, S. ferus, S. gallolyticus, S. gordonii, S. hyointestinalis, S. hyo lis, S. iniae, S. intermedius, S. intestinalis, S. macacae, S. mitis, S. mutans, S. oralis, S. parasanguinis, S. parauberis, S. phocae, S. pleomorphus, S. pneumoniae, S. porcinus, S. pyogenes, S. ratti, S. salivarius, S. sanguinis, S. shiloi, S. sobrinus, S. suis, S. thermophilus, S. thoraltensis, S. uberis, S. vestibularis, and S. viridans.

The new species according to the invention can also be distinguished by this technique from the lactic acid bacteria which had been previously classified in error in the genus Streptococcus such as S. adjacens (new classification=Abiotrophia adiacens), S. casseliflavus (=Eliterococcus casseliflavus), S. cecorum (=Enterococcus cecorum), S. cremoris (=Lactococcus lactis subsp. cremoris), S. defectivus (=Abiotrophia defectiva), S. faecalis (=Enterococcus faecalis), S. faecium (=Enterococcus faecium), S. gallinarum (=Enterococcus gallinarum), S. garvieae (=Lactococcus garvieae), S. hansenii (=Ruminococcus hansenii), S. lactis (=Lactococcus lactis subsp. lactis), S. lactis cremoris (=Lactococcus lactis subsp. cremoris), S. lactis diacetilactis (=Lactococcus lactis subsp. lactis), S. morbillorum (=Gemella morbillorum), S. parvulus (=Atopobium parvulum), S. plantarum (=Lactococcus plantarum), S. raffinolactis (=Lactococcus raffinolactis) and S. saccharolyticus (=Enterococcus saccharolyticus).

The lactic acid bacteria according to the invention have a morphology characteristic of Lactococcus lactis, for example; that is to say that they have the shape of cocci assembled into chains.

The sugars which can be fermented by the new species are generally at least one of the following; D-galactose, D-glucose, D-fructose, D-mannose, N-acetyl-(D)-glucosamine, salicin, cellobiose, maltose, lactose, sucrose or raffinose.

Among all the strains of the new species which have been isolated in dairies in Switzerland, 7 were deposited under the treaty of Budapest, by way of example, in the Collection Nationale de Culture de Microorganisms (CNCM), 25 rue du docteur Roux, 75724 Paris, on 14 Oct. 1997, where they were attributed the deposit numbers CNCM I-1920, I-1921, I-922, I-1923, I-1924, I-1925 and I-1926.

The strains of the new species can be used, for example, to prepare a dietary or pharmaceutical product, in particular in the form of a fresh, concentrated or dried culture.

Milk-based products are obviously preferred within the framework of the invention. Milk is however understood to mean that of animal origin, such as cow, goat, sheep, buffalo, zebra, horse, donkey, or camel, and the like. The milk may be in the native state, a reconstituted milk, a skimmed milk or a milk supplemented with compounds necessary for the growth of the bacteria or for the subsequent processing of fermented milk, such as fat, proteins of a yeast extract, peptone and/or a surfactant, for example. The term milk also applies to what is commonly called vegetable milk, that is to say extracts of plant material which have been treated or otherwise, such as leguminous plants (soya bean, chick pea, lentil and the like) or oilseeds (colza, soya bean, sesame, cotton and the like), which extract contains proteins in solution or in colloidal suspension, which are coagulable by chemical action, by acid fermentation and/or by heat. Finally, the word milk also denotes mixtures of animal and vegetable milks.

Pharmaceutical products means products intended to be administered orally, or even topically, which comprise an acceptable pharmaceutical carrier to which, or onto which, a culture of the new species is added in fresh, concentrated or dried form, for example. These pharmaceutical products may be provided in the form of an ingestible suspension, a gel, a diffuser, a capsule, a hard gelatin capsule, a syrup, or in any other galenic form known to persons skilled in the art.

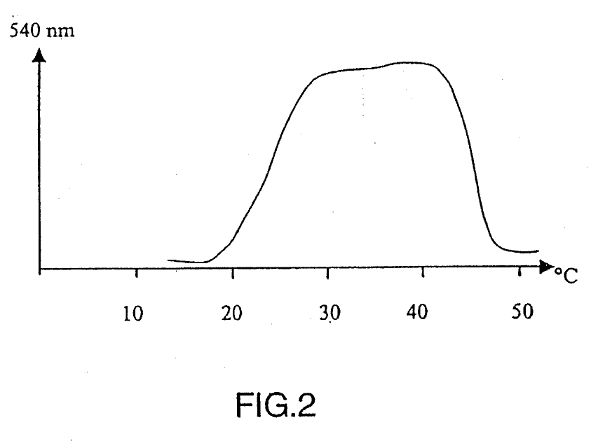

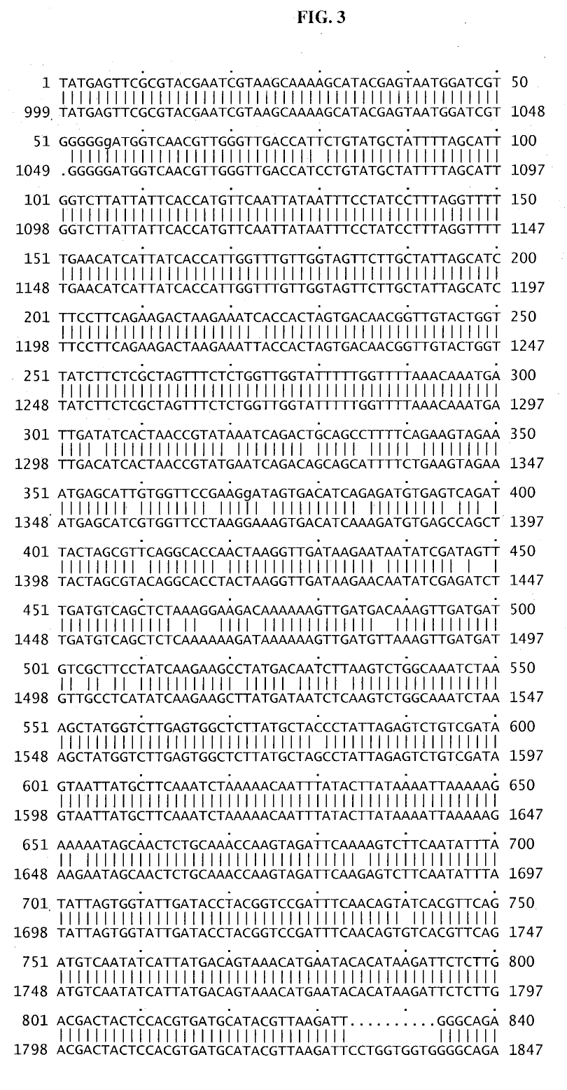

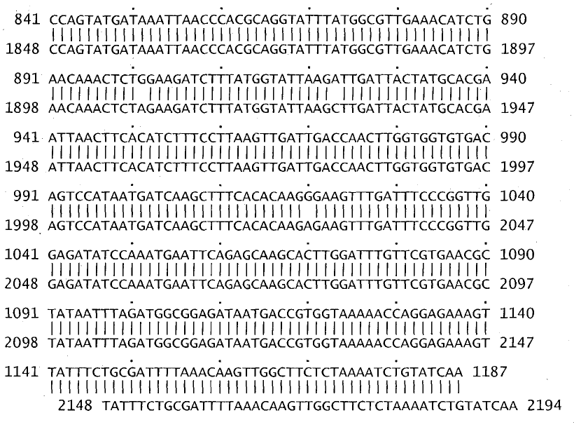

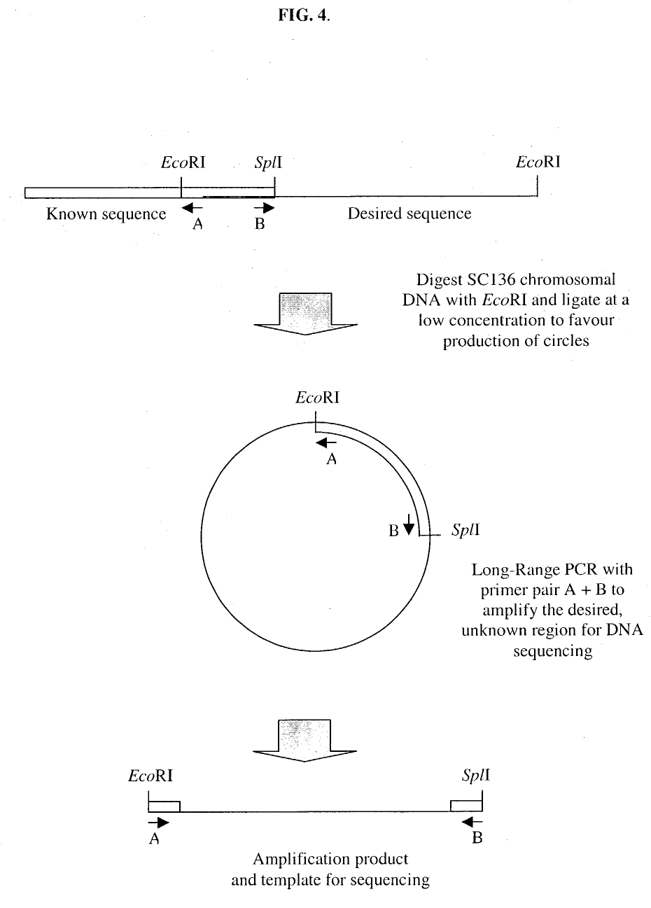

Moreover, some strains of the new species according to the invention, representing a new biotype of this species, may also have the remarkable property of being both mesophilic and thermophilic (mesophilic/thermophilic biotype). The strains belonging to this biotype have a growth optimum from about 28° C. to about 45° C. This property can be easily observed (1) by preparing several cultures of a mesophilic/thermophilic biotype in parallel, at temperatures ranging from 20 to 50° C., (2) by measuring the absorbance values for the media after 16 h of culture, for example, and (3) by grouping the results in the form of a graph representing the absorbance as a function of the temperature (graditherm). FIG. 2 is particularly representative of the graphs, which can be obtained with this type of mesophilic/thermophilic biotype according to the invention. As a guide, among the strains of the new species having this particular biotype, the strains CNCM I-1920, I-1921 and I-1922 are particularly representative, for example.

The use of a mesophilic/thermophilic biotype in the dairy industry is of great importance. Indeed, this species may be used for the preparation of mesophilic or thermophilic starters. It is thus possible to produce industrially acidified milks at 45° C. in order to obtain a “yogurt” type product. It is also possible to industrially produce cream cheese by fermenting a milk in the presence of rennet at 28° C., and separating therefrom the curd thus formed by centrifugation or ultrafiltration. The problems of clogging of the machines linked to the use of thermophilic ferments are thus eliminated (these problems are disclosed in patent application EP No. 96203683.6).

Moreover, other strains of the new species according to the invention, representing another new biotype of this species, may exhibit the remarkable property of conferring viscosity to the fermentation medium (texturing biotype). The viscous character of a milk fermented by a texturing biotype according to the invention may be observed and determined as described below:

1. Comparison of the structure of a milk acidified by a texturing biotype with that of milk acidified by non-texturing cultures; the non-viscous milk adheres to the walls of a glass cup, whereas the viscous milk is self-coherent.

2. Another test may be carried out using a pipette. The pipette is immersed in the acidified milk, which is drawn up in a quantity of about 2 ml, and then the pipette is withdrawn from the milk. The viscous milk forms a rope between the pipette and the liquid surface, whereas the non-viscous milk does not give rise to this phenomenon. When the liquid is released from the pipette, the non-viscous milk forms distinct droplets just like water, whereas the viscous milk forms droplets ending with long strings, which go up to the tip of the pipette.

3. When a test tube filled up to roughly a third of a rotary shaker, the non-viscous milk climbs up the inner surface of the wall, whereas the rise of the viscous milk is about zero.

The viscous character of this particular biotype may also be determined with the aid of a rheological parameter measuring the viscosity. A few commercial apparatus are capable of determining this parameter, such as the rheometer Bohlin VOR (Bohlin GmbH, Germany). In accordance with the manufacturer's instructions, the sample is placed between a plate and a truncated cone of the same diameter (30 mm, angle of 5.4°, gap of 0.1 mm), then the sample is subjected to a continuous rotating shear rate gradient which forces it to flow. The sample, by resisting the strain, develops a tangential force called shear stress. This stress, which is proportional to the flow resistance, is measured by means of a torsion bar. The viscosity of the sample is then determined, for a given shear rate, by the ratio between the shear stress (Pa) and the shear rate (s⁻¹) (see also “Le Technoscope de Biofutur”, May 1997).

The tests of rheological measurement of the texturing character of this biotype have led to the following definition. A lactic acid bacterium belonging to the texturing biotype according to the invention is a bacterium which, when it ferments a semi-skimmed milk at 38° C. up to a pH of 5.2, gives to the medium a viscosity which is greater than 100 mPa.s at a shear rate of the order of 293 s⁻¹, for example. As a guide, the strains CNCM I-1922, I-1923, I-1924, I-1925 and I-1926 are particularly representative of this texturing biotype for example.

This texturing biotype is also of great importance in the dairy industry because its capacity to give viscosity to a dairy product is exceptionally high when it is compared with those of other species of texturing lactic acid bacteria, in particular with the strains Lactobacillus helveticus CNCM I-1449, Streptococcus thermophilus CNCM I-1351, Streptococcus thermophilus CNCM I-1879, Streptococcus thermophilus CNCM I-1590, Lactobacillus bulgaricus CNCM I-800 and Leuconostoc mesenteroides ssp. cremoris CNCM 1-1692, which are mentioned respectively in patent applications EP 699689, EP 638642, EP 97111379.0, EP 750043, EP 367918 and EP 97201628.1.

It is also possible to note that the production of a viscosity may also take place, for some strains, in a very broad temperature range that extends from the mesophilic temperatures (25-30° C.) to the thermophilic temperatures (40-45° C.). This characteristic feature represents an obvious technological advantage.

However, some strains belonging to this new texturing biotype produce an exopolysaccharide (EPS) of high molecular weight whose sugar composition is similar to that found in the oligosaccharides in human breast milk. The EPS in fact consists of a chain of glucose, galactose and N-acetylglucosamine in a proportion of 3:2:1 respectively (A. Kobata, in the Glycoconjugates, Vol. 1, “Milk glycoproteins and oligosaccharides”, p. 423-440, Ed. 1. Horowitz and W. Pigman, Ac. Press, N.Y., 1977). As a guide, the strains CNCM I-1923, I-1924, I-1925 and I-1926 produce this polysaccharide.

This exopolysaccharide, in native or hydrolyzed form, could thus advantageously satisfy a balanced infant diet.

It is possible to prepare a diet for children and/or breast-feeding infants comprising a milk which has been acidified with at least one strain of lactic acid bacterium producing an EPS consisting of a chain of glucose, galactose and N-acetylglucosamine in a proportion of 3:2:1, respectively, in particular with the strains CNCM I-1924, I-1925 or I-1926, for example.

It is also possible to isolate this EPS beforehand from a culture medium of this biotype, and to use it, in native or hydrolyzed form, as an ingredient in an infant diet, for example.

The isolation of the EPS generally consists of removing the proteins and the bacteria from the culture medium and in isolating a purified fraction of the EPS. It is also possible to carry out the extraction of the proteins and of the bacteria by precipitation with an alcohol or trichloroacetic acid followed by centrifugation, while the EPS can be purified by precipitation in a solvent such as acetone followed by centrifugation, for example. If necessary, the EPS may also be purified, for example, by means of gel filtration or affinity chromatography.

In the context of the present invention, the isolation of an EPS also encompasses all the methods of production of an EPS by fermentation followed by concentration of the culture medium by drying or ultrafiltration, for example. The concentration may be performed by any method known to a person skilled in the art, and in particular by freeze-drying or spray-drying in a stream of hot air, for example. To this effect, the methods described in U.S. Pat. No. 3,985,901, EP 298605 and EP 63438 are incorporated by reference into the description of the present invention.

Insofar as the maternal oligosaccharides are small in size, it may be advantageous to carry out beforehand a partial hydrolysis of the EPS according to the invention. Preferably, the hydrolysis conditions are chosen so as to obtain oligosaccharides having 3 to 10 units of sugar, that is to say therefore oligosaccharides having a molecular weight on the order of 600 to 2000 Dalton, for example.

More particularly, it is possible to hydrolyze the EPS according to the invention in a 0.5 N trifluoroacetic acid (TFA) solution for 30-90 min at 100° C., and then to evaporate the TFA and to recover the oligosaccharides.

A preferred infant product comprises hydrolyzed protein material of whey from which allergens, chosen from a group consisting of alpha-lactalbumin, beta-lactoglobulin, serum albumin and the immunoglobulins, have not been removed and in which the hydrolyzed protein material, including the hydrolyzed allergens, exists in the form of hydrolysis residues having a molecular weight not greater than about 10,000 Dalton, such that the hydrolyzed material is substantially free of allergenic proteins and of allergens of protein origin (a hypoallergenic product in accordance with European Directive 96/4/EC; Fritsche et al., Int. Arch. Aller and Appl. Imm., 93, 289-293, 1990).

It is possible to mix the EPS according to the invention, in native or partially hydrolyzed form, with this hydrolyzed protein material of whey, and to then incorporate this mixture, in dried form or otherwise, into numerous food preparations for dietetic use, in particular into foods for infants. EPS can also be mixed with foods intended primarily for people suffering from allergies.

The present invention also relates to the isolated EPS operon (SEQ ID NO:4) and genes thereof, which was isolated from the new species, Streptococcus macedonicus. The present invention also relates to homologues EPS genes, which hybridize with SEQ ID NO:4 or the genes thereof, preferably under highly stringent conditions, e.g., washing in 0.1.times.SSC/0.1% SDS at 68.degree. C. (Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and also any DNA sequence that hybridizes to the complement of the coding sequences disclosed herein under less stringent conditions, such as moderately stringent conditions, e.g., washing in 0.2.times.SSC/0.1% SDS at 42.degree. C. (Ausubel et al., 1989, supra), yet which still encodes a functionally equivalent gene product.

The invention also encompasses DNA vectors that contain any of the coding sequences disclosed herein, and/or their complements (i.e., antisense); DNA expression vectors that contain any of the coding sequences disclosed herein, and/or their complements (i.e., antisense), operatively associated with a regulatory element that directs the expression of the coding and/or antisense sequences; and genetically engineered host cells that contain any of the coding sequences disclosed herein, and/or their complements (i.e., antisense), operatively associated with a regulatory element that directs the expression of the coding and/or antisense sequences in the host cell. Regulatory element includes, but is not limited to, inducible and non-inducible promoters, enhancers, operators and other elements known to those skilled in the art that drive and regulate expression. The invention includes fragments of any of the DNA sequences discussed or disclosed herein.

Standard nucleotide isolation techniques well known to those skilled in the art can be used to isolate the nucleotide sequences disclosed herein or to synthesize them, such as the techniques used in Example 8, as well as suggested primers that can be used.

In another embodiment of the invention the isolated EPS operon and a gene thereof are used in the production of transformed cells having the EPS operon or a gene thereof. Preferably the transformed cell produces an exopolysaccharide, when cultured in milk, comprising a chain of glucose, galactose and N-acetylglucosamine in a proportion of 3:2:1, respectively, characteristic of Streptococcus macedonicus. Production of other exopolysaccharides by the transformed cell are anticipated and encompassed by the present invention however.

Preferably the transformed cell is a microorganism and more preferably a microorganism suitable for use in diary food production suitable for use at temperatures ranging from 20 to 50° C. Transformation/recombination of a nucleic acid molecule into a cell can be accomplished by any method by which a nucleic acid molecule can be inserted into the cell. Transformation techniques include, but are not limited to, transfection, electroporation and microinjection. Preparation and isolation techniques are described by Nelson and Housman, in Gene Transfer (ed. R. Kucherlapati) Plenum Press, 1986.

Recombinant molecules of the present invention, which can be either DNA or RNA, can also contain additional regulatory sequences, such as translation regulatory sequences, origins of replication, and other regulatory sequences that are compatible with the transformed/recombinant cell. One or more recombinant molecules of the present invention can be used to produce an encoded product. A preferred method is by transfecting a host cell with one or more recombinant molecules of the present invention to form a transformed/recombinant cell.

Nucleic acid molecules of the present invention can be operatively linked to expression vectors containing regulatory sequences such as transcription control sequences, translation control sequences, origins of replication, and other regulatory sequences that are compatible with the transformation cell and that control the expression of nucleic acid molecules of the present invention. In particular, recombinant molecules of the present invention include transcription control sequences. Transcription control sequences are sequences, which control the initiation, elongation, and termination of transcription. Particularly important transcription control sequences are those that control transcription initiation, such as promoter, enhancer, operator and repressor sequences. Suitable transcription control sequences include any transcription control sequence that can function in yeast or bacterial cells. A variety of such transcription control sequences are known to those skilled in the art.

It may be appreciated by one skilled in the art that use of transformation DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Transformation techniques useful for increasing the expression of nucleic acid molecules of the present invention include, but are not limited to, operatively linking nucleic acid molecules to high-copy number plasmids, integration of the nucleic acid molecules into the host cell chromosome, addition of vector stability sequences to plasmids, substitutions or modifications of transcription control signals (e.g., promoters, operators, enhancers), substitutions or modifications of translational control signals, modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

Additional identifying characteristics of the new species have now been identified recently. (See Schlegel, L.; Grimont, F.; Ageron, E.; Grimont, P.; and Bouvet, A. (2003) “Reappraisal of the taxonomy of the Streptococcus bovis/Streptococcus equinus complex and related species: description of Streptococcus gallolyticus subsp. gallolyticus subsp. nov., S. gallolyticus subsp. macedonicus subsp. nov. and S. gallolyticus subsp. pasteurianus subsp. nov. ” Int. J. Syst. Evol. microbial. 53(3), 631-645). These identifying characteristics include additional phenotypic characterizations.

Phenotypic characterization of the new species, Streptococcus macedonicus typically include the following characteristics: gram-positive cocci, non-motile, and non-sporulating. The catalase test is typically negative. The strains of this species generally show homogeneous growth in buffered glucose and brain heart infusion broths and do not produce gas in MRS broth. Typically they are non-haemolytic on sheep-blood agar in an aerobic atmosphere and are tellurite-negative. The strains generally produce leucine aminopeptidase and alanyl-phenylalanyl-proline arylamidase and do not produce—glucuronidase. Further phenotypic characterizations of the new species include production of galactosidase (−GAR test) and usually negative for—glucosidase. They usually do not hydrolyze aesculin and do not typically produce acid from glycogen or inulin, or produce tannase. They do not produce acid from melibiose. Production of acid from methyl-D-glucopyranoside and starch is variable. One type strain of the new species is ACA-DC 206T (=LAB 617T=ATCC BAA-249T=CCUG 39970T=CIP 105683T=JCM 11119T=LMG 18488T=HDP 98362T).

Quantitative DNA-DNA hybridization relatedness test can be determined by labeling the DNA in vitro with [3H]ATP, [3H]TTP, [3H]GTP and [3H]CTP using the Megaprime DNA labelling reaction kit (all from Amersham). Hybridizations of these labelled DNAs with DNA of representative strains of the S. macedonicus, preferable CNCM I-1920, I-1921, I-1922, I-1923, I-1924, I-1925 or I-1926, and more preferable CNCM I 1923 or I-1924. Preferably the hybridization complex is carried out in a liquid medium under stringent conditions consisting of 60 ° C. for 16 h, according to a modification of the S1 nuclease/trichloracetic acid precipitation method (Crosa et al., 1973; Grimont et al., 1980). The temperature at which 50% of the reassociated DNAs were hydrolysed by S1 nuclease (Tm) is determined. The difference between the melting temperatures of homoduplexes and heteroduplexes (Tm) is one method of determining DNA divergence between strains with high levels of DNA relatedness (Grimont et al., 1980).

16S rDNA sequence determination relatedness can be determined by aligning the sequences using the CLUSTAL multiple-sequence method. A distance matrix can then computed using a Kimura model for nucleotide substitution. Alignment with a selection of the available sequences of 16S rDNA characteristic of Streptococcus genus, from GenBank and phylogenetic analysis of the 16S rDNA data can be performed with the MEGALIGN program from the DNAstar package.

The present invention is described in greater detail by the examples presented below. It goes without saying however, that these examples are given by way of illustration of the subject of the invention and do not constitute in any manner a limitation thereto. The percentages are given by weight unless otherwise stated.

### EXAMPLES

### Example 1

Identification of a New Species of Streptococcus

Several strains of lactic acid bacteria isolated from various dairies in Switzerland were the subject of the following genetic and physiological identification. The methods used as well as the results obtained, which are represented below, show that these strains are part of a new Streptococcus group which is sufficiently distinct and homogeneous for it to be designated as grouping together a new species of lactic acid bacterium. By way of example, some strains belonging to this new species were deposited under the treaty of Budapest in the Collection Nationale de Culture de Microorganismes (CNCM), 25 rue du docteur Roux, 75724 Paris, on 14 Oct. 1997, where they received the identification Nos. CNCM I-1920, I-1921, I-1922, I-1923, I-1924, I-1925 and I-1926.

1. Morphology of the strains isolated: A morphology characteristic of Lactococcus lactis, that is to say a shape of cocci assembled into chains, was observed under a microscope.

2. Sugar fermentation profile of the strains isolated: The sugars which can be fermented by the isolated strains are generally D-galactose, D-glucose, D-fructose, D-mannose, N-acetyl-(D)-glucosamine, salicin, cellobiose, maltose, lactose, sucrose and raffinose. This fermentation profile was similar to that obtained with the species Lactococcus lactis.

3. 16S ribosomal RNA of the strains isolated: The isolated strains were cultured in 40 ml of HJL medium at 37° C. for 24 h, the bacteria were harvested by centrifugation, each bacterial pellet was resuspended in 2.5 ml of TE buffer (10 mM Tris PH 8, 0.1 mM EDTA) containing 10 mg/ml of lysozyme, and the whole was incubated at 37° C. for 1 h. 100 μl of a solution containing 10 mg/ml of proteinase K, 250 μl of a solution containing 500 mM EDTA pH 8.0, and 500 μl of a solution containing 10% SDS was then added. The whole was incubated at 60° C. for 1 h so as to ensure complete lysis of the bacteria. After having cooled the mixtures, 2.5 ml of phenol/chloroform was added, and they were centrifuged for 10 min in a Heraeus centrifuge so as to separate 2 phases. The top phase was removed. The chromosomal DNA present in the bottom phase was precipitated by addition of 2.5 ml of a solution containing 96% ethanol, and the mixture was gently stirred until a precipitate was formed. The precipitated DNA was removed with the aid of a wooden toothpick, deposited in a 2 ml Eppendorf tube containing 1 ml of a Tris buffer (10 mM Tris HCl pH 8.0, 10 mM EDTA and 10 μg/ml of RNase A), and incubated at 56° C. for 1 h. After cooling, the various suspensions of DNA were extracted with 1 ml of phenol/chloroform as described above, and the chromosomal DNA was precipitated with ethanol. The DNA was resuspended in an Eppendorf tube containing a quantity of TE buffer such that the final quantity of DNA for each strain isolated was about 250 μg/ml.

An aliquot of 1 μl of DNA of each strain isolated was amplified by PCR with the primers having the respective nucleotide sequences SEQ ID NO:2 and SEQ ID NO:3 (see sequence listing), for 30 cycles (95° C./30 sec, 40° C./30 sec and 72° C./2 min) using Pwo polymerase from Boehringer. The PCR products were purified with the aid of the QIAGEN QIAquick kit, and the products were eluted in 50 μl of TE buffer. A sample of 20 μl of each product was digested with the restriction enzymes BamHI and SalI, and the 1.6 kb fragments were separated on an agarose gel (1%), and purified with the aid of the QIAGEN QIAquick kit. The fragments were then cloned into the E. coli vector pK19 (R. D. Pridmore, Gene 56, 309-312, 1987) previously digested with BamHI and SalI and dephosphorylated, and competent cells of E. coli strain BZ234 (University of Basel collection, Switzerland) were transformed with each ligation product. The transformants were selected for at 37° C. on LB medium with 50 μg/ml of kanamycin, 30 ng/ml of X-gal and 10 ng/ml of IPTG. The white colonies containing the insert were cultured for 10 h on LB medium with 50 μl/ml of kanamycin, and the plasmid DNAs were isolated with the aid of the QIAGEN QIAprep8 kit.

A 4 μl sample of each plasmid (1 pmol/μl: obtained from each strain isolated) were mixed with 4 μl of labelled primers IRD-41 (sequencing primers: MWG Biotech) and 17 μl of H₂O. For each strain isolated, 4 aliquots of 6 μl were added to 4 wells of 200 μl, and 2 μl of a reaction mixture (Amersham; RPN2536) was then added to the wells. The mixtures were amplified by PCR in the Hybaid Omn-E system with 1 cycle of 95° C. for 2 min followed by 25 cycles of 95° C./30 sec, 50° C./30 sec and 72° C./1 min. The reaction products were then separated conventionally on a polyacrylamide gel, and the DNA sequence was determined for each isolated strain. The DNA fragments thus sequenced represented the genomic part of the 16S ribosomal RNA.

The results show that all the strains isolated contain a nucleotide sequence similar, or even identical, to the sequence identified in SEQ ID NO:1 which is disclosed in the sequence listing. These sequences exhibit numerous homologies with the 16S RNA sequences found in the species of lactic acid bacteria belonging to the genus Streptococcus, which leads to these strains being classified in the genus Streptococcus. For example, Streptococcus thermophilus 95% ID, Lactobacillus Lactis 89% ID, Lactobacillus bulgaricus 88% ID, Lactobacillus Helveticus 84% ID, and Lactobacillus Johnsonii 86% ID.

4. Identification by SDS-PAGE electrophoresis gel: The tests were carried out in accordance with the instructions provided by Pot et al., presented during a “workshop” organized by the European Union, at the University of Ghent, in Belgium, on 12 to 16 Sep. 1994 (fingerprinting techniques for classification and identification of bacteria, SDS-PAGE of whole cell protein).

In short, to cultivate the lactic acid bacteria, 10 ml of MRS medium (of Man, Rogosa and Sharpe) are inoculated with an MRS preculture of each strain of the new species of lactic acid bacterium, as well as of each reference strain covering as many species of Streptococcus as possible. The media are incubated for 24 h at 28° C., they are plated on a Petri dish comprising a fresh MRS-agar medium, and the dishes are incubated for 24 h at 28° C.

To prepare the extract containing the proteins of the bacteria, the MRS-agar medium is covered with a pH 7.3 buffer containing 0.008 M of Na₂HPO₄.12H₂O, 0.002 M of Na₂HPO₄.2H₂O and 8% NaCl. The bacteria are recovered by scraping the surface of the gelled medium, the suspension is filtered through a nylon gauze, it is centrifuged for 10 min at 9000 rpm with a GSA rotor, the pellet is recovered and taken up in 1 ml of the preceding buffer. The pellet is washed by repeating the centrifugation-washing procedure, finally about 50 mg of cells are recovered to which one volume of STB buffer pH 6.8 (per 1000 ml: 0.75 g Tris, 5 ml C₂H₆OS, 5 g of glycerol) is added, the cells are broken by ultrasound (Labsonic 2000), the cellular debris is centrifuged, and the supernatent containing the total protein is preserved.

An SDS-PAGE polyacrylamide gel 1.5 mm thick (Biorad-Protean or Hoefer SE600), crosslinked with 12% acrylamide in the case of the separating gel (12.6 cm in height) and 5% acrylamide in the case of the stacking gel (1.4 cm in height), is then conventionally prepared. For that, the polymerization of the two gel parts is carried out in particular in a thermostated bath at 19° C. for 24 h and 1 h respectively, so as to reduce the gel imperfections as much as possible and to maximize the reproducibility of the tests.

The proteins of each extract are then separated on the SDS-PAGE electrophoresis gel. For that, 6 mA are applied for each plate containing 20 lanes until the dye reaches a distance of 9.5 cm from the top of the separating gel. The proteins are then fixed in the gel, they are stained, the gel is dried on a cellophane, the gel is digitized by means of a densitometer (LKB Ultroscan Laser Densitometer, Sweden) linked to a computer, and the profiles are compared with each other by means of the GelCompar® software, version 4.0, Applied Maths, Kortrijk, Belgium. Insofar as the tests were sufficiently standardized, the profiles of the various species of Streptococcus contained in a given library were also used during the digital comparison.

The results then show that all the strains tested belonging to the new species can be distinguished from all of the following species: S. acidominimus, S. adjacens, S. agalactiae, S. alactolyticus, S. anginosus, S. bovis, S. canis, S. caprinus, S. casseliflavus, S. cecorum, S. constellatus, S. cremoris, S. cricetus, S. cristatus, S. defectivus, S. difficile, S. downei, S. dysgalactiae ssp. dysgalactiae, S. dysgalactiae ssp. equisimilis, S. equi, S. equi ssp. equi, S. equi ssp. zooepidemicus, S. equinus, S. faecalis, S. faecium, S. ferus, S. gallinarum, S. gallolyticus, S. garvieae, S. gordonii, S. hansenii, S. hyointestinalis, S. hyo lis, S. iniae, S. intermedius, S. intestinalis, S. lactis, S. lactis cremoris, S. lactis diacetilactis, S. macacae, S. mitis, S. morbillorum, S. mutans, S. oralis, S. parasanguinis, S. parauberis, S. parvulus, S. phocae, S. plantarum, S. pleomorphus, S. pnemoniae, S. porcinus, S. pyogenes, S. raffinolactis, S. ratti, S. saccharolyticus, S. salivarius, S. sanguinis, S. shiloi, S. sobrinus, S. suis, S. thermophilus, S. thoraltensis, S. uberis, S. vestibularis and S. viridans.

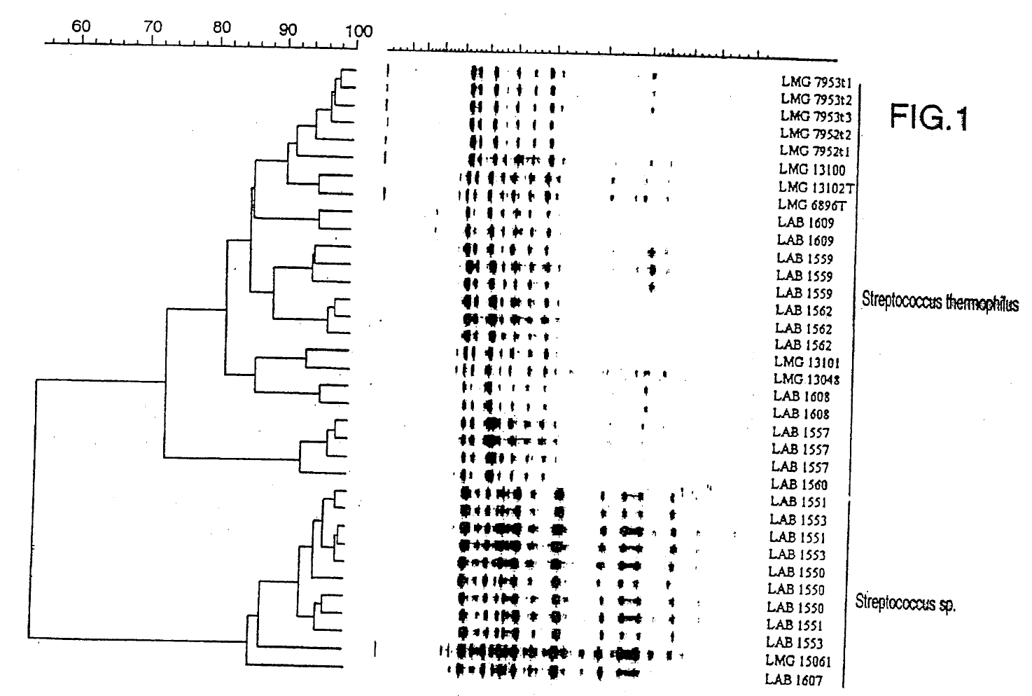

All the results show that the degree of Pearson correlation between the strains deposited is at least 85. As a guide, FIG. 1 depicts a photograph of one of the electrophoresis gels, the filiation in the form of a tree, as well as the degree of Pearson correlation (indicated on the top left-hand scale). The strains LAB 1550, LAB 1551 and LAB 1553 refer specifically to the strains CNCM I-1921, I-1922 and I-1925. The strains LMG15061 and LAB 1607 were not deposited at the CNCM, but obviously form part of this new species.

In short, all the strains isolated clearly form part of a homogeneous group, which is distinct from the other species belonging to the genus Streptococcus.

### Example 2

Mesophilic/Thermophilic Biotype

Some strains isolated in Example 1 represent a new particular biotype since they exhibit the remarkable property of being both mesophilic and thermophilic.

This property may easily be observed (1) by preparing, in parallel, several cultures of a mesophilic/thermophilic biotype in an M17-lactose medium at temperatures ranging from 20 to 50° C., (2) by measuring the absorbance values for the media at 540 nm after 16 h of culture, and (3) by grouping the results in the form of a graph representing the absorbance as a function of the temperature (graditherm).

FIG. 2 represents the graditherm obtained with the strain CNCM I-1920. All the other strains isolated belonging to this particular biotype, in particular the strains CNCM I-1921 and I-1922, also give comparable graditherms.

### Example 3

Texturing Biotype

Several strains isolated in Example 1 had the remarkable property of being extremely texturing. This property was observed with the aid of the rheological parameter of viscosity measured with a Bohlin VOR rotational rheometer (Bohlin GmbH, Germany).

For that, some of the strains isolated were cultured in a semi-skimmed milk at 38° C. with a pH up to about 5.2. In accordance with the manufacturer's instructions, a sample of each culture medium was then placed between a plate and a truncated cone of the same diameter (30 mm, angle of 5.4□, gap of 0.1 mm), then the sample was subjected to a continuous rotating shear rate gradient which forces it to flow. The viscosity of the sample was then determined at a shear rate of 293⁻¹. The results of the rheology tests carried out with some of the strains isolated demonstrated that the culture media thus fermented had a viscosity greater than 100 mPa.s, or even a viscosity exceeding 200 mPa.s in the case of the strains CNCM I-1922, I-1923, I-1924, I-1925 and I-1926.

For comparison, viscosities of the order of 54, 94, 104, 158 and 165 mPa.s were obtained, under the same operating conditions, with the strains Lactobacillus helveticus CNCM I-1449, Streptococcus thermophilus CNCM I-1351, Streptococcus thermophilus CNCM I-1879, Streptococcus thermophilus CNCM I-1590, Lactobacillus bulgaricus CNCM I-800 and Leuconostoc mesenteroides ssp. cremoris CNCM I-1692, respectively, which were mentioned in patent applications EP 699689, EP 638642, EP 97111379.0, EP 750043, EP 367918 and EP 97201628.1, respectively (the strains CNCM I-800 and I-1692 were reputed to be highly texturing strains).

### Example 4

New Exopolysaccharide

Some strains isolated in Example 1, belonging to the texturing biotype, in particular the strains CNCM I-1923, I-1924, I-1925 and I-1926, produced an EPS of high molecular weight whose sugar composition was similar to those found in certain oligosaccharides in human breast milk. Analysis of the sugars constituting this polysaccharide was carried out in the following manner.

The strains of the new species were cultured in 10% reconstituted skimmed milk, with shaking, for 24 h at 30° C., the pH being maintained at 5.5 by addition of a 2 N NaOH solution. The bacterial cells and the proteins were removed from the culture medium by means of precipitation in an equal volume of a solution of 25% by weight of trichloroacetic acid, followed by centrifugation (10,000 g, 1 h). The EPSs were precipitated by addition of an equivalent volume of acetone, followed by settling for 20 h at 4° C. The EPSs were recovered by centrifugation, and the pellet was taken up in a 0.1 M NH₄HCO₃ solution pH 7, and the suspension was dialyzed against water for 24 h. The insoluble materials were then removed by ultracentrifugation, and the retentate containing the purified EPS was freeze-dried. The quantity of purified EPS, expressed as mg of glucose equivalent, was on the order of 40 mg per liter of culture.

The molecular weight of the EPS was determined by means of gel-filtration chromatography with the aid of a Superose-6 column connected to an FPLC system (Pharmacia), as described by Stingele et al., J. Bacteriol., 178, 1680-1690, 1996. The results demonstrated that all the strains CNCM I-1923, I-1924, I-1925 and I-1926 produce an EPS of a size greater than 2×10⁶ Da.

100 mg glucose equivalent of the purified EPS was hydrolyzed in 4 N TFA at 125° C. for 1 h, before being derivatized and analyzed by GLC chromatography according to the method described by Neeser et al. (Anal. Biochem., 142, 58-67, 1984). The results demonstrated that the strains produced an EPS consisting of glucose, galactose and N-acetylglucosamine in a mean proportion of 3:2:1, respectively.

### Example 5

Infant Product

A whey, 18% hydrolyzed with trypsine is prepared according to the recommendations of U.S. Pat. No. 5,039,532. It is traditionally spray-dried in a stream of hot air, and between 0.1 and 10% of the dry purified EPS described in Example 4 is incorporated into it. This product can be rapidly reconstituted in water. It is particularly suitable for a diet for children or breast-feeding infants because of its hypoallergenic and tolerogenic properties to cow's milk, and because it is balanced from a carbohydrate composition point of view.

### Example 6

Infant Product

The dry purified EPS of Example 4 is hydrolyzed in a 0.5 N trifluoroacetic acid (TFA) solution for 30-90 min and at 100° C., the TFA is evaporated, the hydrolyzate is suspended in water and the oligosaccharides having 3 to 10 units of sugar (600 to 2000 Dalton) are separated by ultrafiltration.

A whey, 18% hydrolyzed with trypsine is prepared according to the recommendations of U.S. Pat. No. 5,039,532. It is traditionally spray-dried in a stream of hot air, and between 0.1 and 10% of purified oligosaccharides described above is incorporated into it. This product can be rapidly reconstituted in water. It is particularly suitable for a diet for children or breast-feeding infants because of its hypoallergenic and tolerogenic properties to cow's milk, and because it is balanced from a carbohydrate composition point of view.

### Example 7

Pharmaceutical Product

A pharmaceutical composition is prepared in the form of a capsule manufactured based on gelatin and water, and which contains 5 to 50 mg of the purified EPS of Example 4 or the purified oligosaccharides of Example 6.

An alternative pharmaceutical product is a pastille consisting of a culture of the freeze-dried strain CNCM I-1924 are prepared and then compressed with a suitable binding agent. These pastilles are particularly recommended for restoring intestinal flora of lactic acid bacteria and for satisfying a balanced diet in terms of essential complex carbohydrates.

### Example 8

Isolation and Analysis of the Streptococcus macedonicus Exopolysaccharide Synthesis (EPS) Operon and the Genes thereof

The Streptococcus macedonicus exopolysaccharide synthesis (EPS) operon was identified, cloned and sequenced as described below. Bioinformatic analysis confirmed the presence of numerous genes related to established exopolysaccharide production in both food-grade and some pathogenic Streptococcus species. Based on the derived DNA sequence and the associated bioinformatic analysis, the EPS operon of the new species, S. macedonicus responsible for production of a unique exopolysaccharide was identified and isolated as described herein.

An interesting property of S. macedonicus is its ability to produce and secrete a polysaccharide with interesting texturing properties and a sugar composition that indicates a potential use in infant and medical applications. The exopolysaccharide composition of glucose, galactose and N-acetylglucosamine in a ratio of 3:2:1 is similar to the sugar composition of maternal milk and would satisfy a well-balanced diet for infant nutrition. The S. macedonicus strain CNCM I-1923 exopolysaccharide has a branched structure with a repeating three sugar backbone and a three sugar side-chain. The oligosaccharide repeating unit structure has been determined and is shown here:

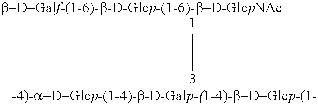

Exopolysaccharides are produced by a variety of microorganisms where they may have diverse functions. In the pathogenic bacterium Streptococcus pneumoniae, the capsular polysaccharide coats the surface of the bacterium and protects it from the environment and host defense mechanisms. The importance of the capsule polysaccharide is seen in S. pneumoniae strains devoid of capsule polysaccharide production which are no longer virulent, while harmless strains producing the capsule polysaccharides at the surface are able to induce the production of protective antibodies by the host. In food-grade lactic acid bacteria, the biological advantage of exopolysaccharide production is less well understood. The present invention provides for use of these exopolysaccharides as natural texturing agents in certain foods.

Three mechanisms have so far been elucidated for the secretion and assembly of exopolysaccharides in bacteria. In the first pathway, as determined for the O-antigen of Salmonella enterica (Reeves P. (1994) Biosynthesis and assembly of lipopolysaccharide, in Bacterial Cell Wall. Ghyusen J.-M. and Hakenbeck R. (eds). Amsterdam: Elsevier Science, pp 281-317), the repeat units are individually synthesized by sequential transfer of sugars by the transferases onto a lipid carrier in the cytoplasm. The units are then transferred to the periplasmic face where polymerization occurs. In the second pathway, as determined for the O-antigen of Escherichia coli O9, N-actetylglucosamine is transferred to undecaprenol phosphate which then serves as the acceptor molecule for the addition of the sugars (see Kido N., Torgov V. I., Sugiyama T., Uchiya K., Sugihara H., Komatsu T., Kato N. and Jann K. (1995). Expression of the O9 polysaccharide of Escherichia coli: sequencing of the E. coli O9 rfb gene cluster, characterization of mannosyl transferases, and evidence for an ATP-binding cassette transport system. J. Bacteriol. 177:2178-2187). However, the N-actetylglucosamine is removed before polymerization and is therefore not a component of the final polysaccharide. Secretion and polymerization are similar to the first example. In the third pathway, as determined for Salmonella enterica serovar Borreze (Keenleyside W. J. and Whitfield C. (1996) A novel pathway for O-polysaccharide biosynthesis in Salmonella enterica serovar Borreze. J. Biol. Chem. 271:28581-28592), initiation is as for the second pathway, but secretion does not use a transporter. Instead, the processive glycosyltransferase may couple the polymerization of the chain to its transport through a pore-like structure in the membrane.

DNA fragments were cloned or generated by PCR for sequence determination and bioinformatic analysis. A single operon of approximately 17 Kb pairs containing the essential genetic elements for the production and secretion of the polysaccharide was identified.

Materials and Methods

1.1 Strains and Grouth Conditions

The bacterium Streptococcus macedonicus CNCM I-1923 Institue Pasteur Collection (also known as NCC2419 and Sc136 strain) and I-1926 from Belgian Culture Collection (NCC1965, Sc147 strain) were provided by the Nestlé Culture Collection and cultivated in HJL medium at 37° C. The laboratory Escherichia coli strain XL1-blue (Stratagene Corp. genotype: recA1 endA1 gyrA96 thi-1 hsdR17 supE44 relA1 lac [F′ proAB lacI^{q}ZΔM15 Tn10]) used for all cloning experiments was cultivated in LB medium at 37° C. with vigorous shaking.

1.2 Chromosomal DNA Preparation from CNCM I-1923

Total DNA was prepared from S. macedonicus strain CNCM I-1923 for cloning and sequencing from a 40 ml culture as follows. A 24 hr culture of CNCM I-1923 in HJL medium grown at 37° C. was centrifuged to recover the bacteria. The cell pellet was suspended in 2.5 ml of TE buffer (10 mM Tris pH 8.0, 1 mM EDTA) containing 10 mg/ml lysozyme and incubated at 37° C. for one h. 100 μl of a 10 mg/ml proteinase K solution, 250 μl of 500 mM EDTA pH8.0 and 500 μl 10% SDS were added and the solution gently mixed and incubated at 60° C. for one h. After cooling, the mixture was extracted once with 2.5 ml of phenol/chloroform mixture, centrifuged at 3,000 rpm to separate the phases and the upper phase removed to a clean tube. The DNA was precipitated by adding 6 ml of 95% ethanol with gentle mixing and transferred to a clean tube with a sterile toothpick. Two ml of a solution of 10 mM Tris-HCl pH8.0, 10 mM EDTA and 10 μg/ml RNase A was added to the DNA and incubated at 60° C. for one h. After cooling, the solution was extracted once with one ml of phenol/chloroform and the chromosomal DNA again precipitated. This final DNA pellet was suspended in TE buffer to give a final concentration of approximately 500 μg/ml.

1.3 Transformation of E. coli

A fresh over-night culture of XL1-blue was used to inoculate 100 ml of LB medium at 1%. This was incubated at 37° C. with vigorous shaking until an OD600 of 1.0 was reached. At this point, the bacteria were recovered from the culture by centrifugation at 8,000 rpm for 10 min in a GSA rotor and a Sorvall HB3 centrifuge. The culture supernatant was discarded and the bacteria suspended in 100 ml of sterile water at 4° C. The bacteria were recovered by centrifugation and the wash repeated a total of three times. The bacteria were finally suspended in 2 ml of sterile 10% glycerol and frozen at −80° C. in convenient aliquots.

Electro-transformation was performed using a BIO-RAD Gene Pulser® with Pulse Controller, 0.2 cm cuvettes and a single pulse of 2,500 V, 25 μFD and 200 Ω. The bacteria were removed in 500 μl of LB medium and incubated at 37° C. with shaking before plating.

1.4 Cloning and DNA Sequence Determination of the Eps Operon

1.4.1 EspA Gene Cloning

The DNA sequence of the regulatory gene epsA from S. thermophilus Sfi6 (Stingele F., Nesser J.-R. and Mollet B. (1996) Identification and characterization of the eps (exopolysaccharide) gene cluster from Streptococcus thermophilus Sfi6. J. Bacteriol. 178:1680-1690), was used to design the PCR primer pair 6143 (^{5′}ATGAGTTCGCGTACGAATCG^{3′}) (SEQ ID NO:7) and 6144 (^{5′}ATACAGATTTTAGAGAAGCC^{3′}) (SEQ ID NO:8). The amplification reaction contained one μl CNCM I-1923 chromosomal DNA (500 ng), 6 μl of 2 mM dNTPs, 2 μl of oligo of each oligonucleotide at 100 nM/ml, 10 μl 10×SuperTaq reaction buffer, 80 μl H₂O and 0.3 μl SuperTaq DNA polymerase in a 0.5 ml PCR tube. PCR was performed in a Perkin-Elmer DNA Thermal Cycler with 30 cycles of 95° C. for 30 sec, 50° C. for 30 sec, 72° C. for 3 min and finally held at 4° C. The PCR reaction was electrophoresed on a 1% agarose gel and an amplification product of approximately 1.2 kb visualised. This amplicon was cut out of the gel, the DNA eluted using the QIAquick gel extraction kit (QIAgen, Product number 28704) and ligated into the vector pGem®-T Easy vector system 1 (Promega, Product number A1360). After electro-transformation into E. coli strain XL1-blue, transformants were selected on LB plates supplemented with 100 μg/ml ampicillin, 300 ng/ml X-gal (5-bromo-4-chloro-3-indolyl-β-D-galactopyranoside, Roche Molecular Biochemicals product number 651 745) and 60 ng/ml IPTG (isopropyl-β-D-thiogalactoside, Roche Molecular Biochemicals product number 724 815) at 37° C. White colonies, which have a high probability of containing DNA inserts, were grown in small-scale 3 ml cultures and the plasmid DNA extracted using the QIAprep 8 Miniprep kit (QIAgen, Product number 27144). Samples of extracted plasmid were digested with the restriction enzyme SacI to identify plasmids containing inserts of the expected size. A plasmid was chosen and named pGem-T/epsA.

1.4.2 Sequencing of pGem-T/epsA

The plasmid pGem-T/epsA was sequenced using the IRD800 labeled fluorescent forward (5′CTGCAAGGCGATTAAGTTGGG^{3′}) (SEQ ID NO:9) and the reverse (^{5′}GTTGTGTGGAATTGTGAGCGG^{3′}) (SEQ ID NO:10) primers and the Thermo Sequenase fluorescent labeled primer cycle sequencing kit with 7-deaza-dGTP (Amersham Pharmacia, RPN2538). The cycle sequencing was performed on the HyBaid Omn-E PCR machine with a single incubation at 95° C. for 5 min, followed by 25 cycles of 95° C. for 30 sec, 50° C. for 72° C. for 2 min and finally held at room temperature. After cycle sequencing, the sequences were electrophoresed and analyzed on the LiCor DNA sequencer. The DNA sequences were exported to the GCG suite of programs for analysis.

1.4.3 Cloning and Identification of pK19-CNCM I-1923/eps

Using the above sequence information, a clone bank of CNCM I-1923 SplI chromosomal DNA fragments was produced and screened by PCR for larger clones containing the epsA homologue. Three μg of chromosomal DNA were digested to completion with the restriction enzyme SplI. A 300 ng sample was ligated into the E. coli vector pK19, previously digested with the restriction enzyme Asp718, an enzyme that produces a 4 base-pair 5′ overhang that is compatible with that generated by SplI. (See Pridmore R. D. (1987) New and versatile cloning vectors with kanamycin-resistance marker. Gene 56:309-312) The ligation mixture was electro-transformed into frozen competent XL1-blue, plated onto LB plates supplemented with 50 μg/ml kanamycin, Xgal and IPTG and incubated at 37° C. for 16 hr. White colonies were tooth-picked into 200 μl volumes of LB medium supplemented with 50 μg/ml kanamycin in microplates and incubated at 37° C. to produce mini-cultures. Five microplates of cultures were produced. 20 μl samples were taken from each of the 12 wells in a row and pooled into a single microtube. A one μl sample from each pool was screened by PCR with the primer pair 6143 and 6144 using the conditions described above. Samples of the PCR reactions were visualised on a 1.5% agarose gel, a PCR positive pool identified and the PCR detection repeated on the 12 individual wells. The bacteria from the PCR positive well were used to inoculate a culture in LB medium supplemented with kanamycin for plasmid isolation and restriction enzyme mapping and DNA sequence determination.

1.4.4 Inverted PCR

The inverted PCR technique was used to prepare template DNA fragments flanking the SplI clone for DNA sequence analysis. In this technique, chromosomal DNA is digested with frequently cutting restriction enzymes and ligated in conditions favoring the formation of circular products. These circles were then used as template for the PCR reaction with appropriately designed PCR primer pairs. Three μg of CNCM I-1923 chromosomal DNA was digested to completion with the restriction enzymes EcoRI, HindIII, NsiI and BclI. These digested DNAs were then phenol extracted, ethanol precipitated and ligated in a 400 μl volume with 10 units T4 DNA ligase (Roche Molecular Biochemicals, Product number 716 359) at 20° C. for 16 h. The ligations were finally phenol extracted, ethanol precipitated and dissolved in 50 μl TE buffer. One μl of the above inverted PCR template was then used as a template for long-range PCR using primer pairs designed according to the strategy shown in FIG. 4 and the Expand PCR kit (Roche Molecular Biochemicals, Product number 1 681 842) according to the provided instructions. The total PCR reaction was electrophoresed on a preparative 1% agarose gel, strong PCR products cut out, the DNA eluted and used as a template for DNA sequencing using custom labeled primers.

1.4.5 Confirmation of the DNA Sequence

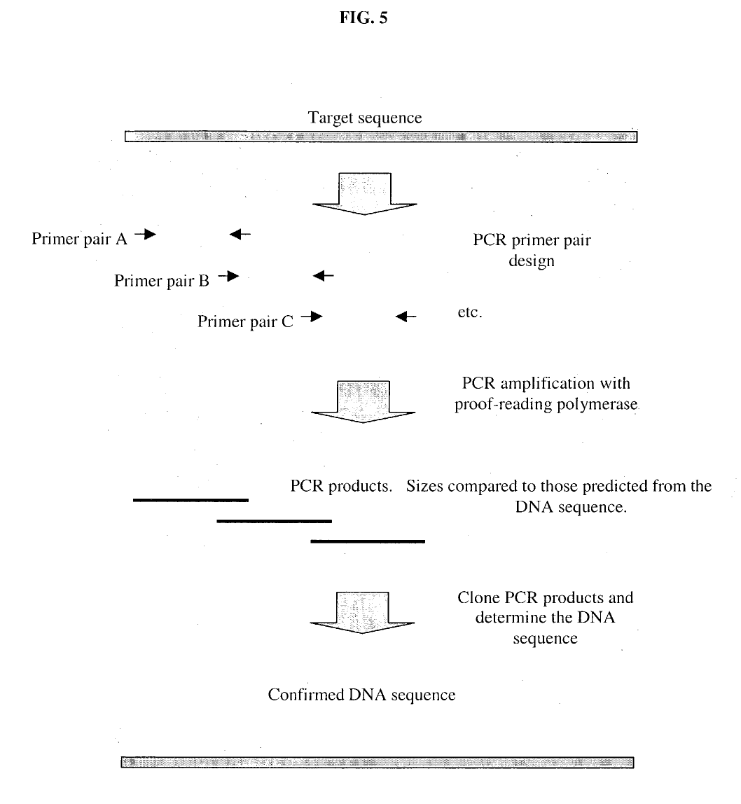

Due to the possibility of rearrangements of foreign DNA when cloned in high copy-number plasmids (such as pK19) in E. coli and the use of inverted PCR products as sequencing templates, the integrity of the DNA sequence was confirmed by the PCR strategy outlined in FIG. 5. PCR primer pairs were designed to amplify approximately 1200 base-pair fragments directly from the CNCM I-1923 genomic DNA. The primer pairs were also positioned so that the amplified fragments overlapped by approximately 200 base-pairs and in this way completely covered the region of interest on both strands. The proof-reading thermostable polymerase Pwo (Roche Molecular Biochemicals, product number 1 644 947) was used to amplify the fragments from CNCM I-1923 chromosomal DNA, the fragments visualised on a 1.0 % agarose gel and compared to the predicted sizes.

The PCR amplicons were purified using the QIAquick PCR cleanup kit (Qiagen, Product number 28104), digested with the restriction enzymes KpnI and BamHI and the DNA fragments resolved of a preparative one % agarose gel. The corresponding bands were cut out of the gel and the DNA eluted using the QIAquick gel extraction kit. These DNA fragments were ligated into the vector pK19, previously digested with the restriction enzymes BamHI and KpnI and dephosphorylated. The ligation was electro-transformed into competent XL1-blue, 500 μl of LB medium added and the transformed bacteria incubated at 37° C. for 90 min. Aliquots of transformed cells were plated onto LB plates supplemented with kanamycin, X-gal and IPTG and incubated at 37° C. for 16 h. Small-scale plasmid preparations were made from a selection of white colonies and subjected to restriction enzyme analysis. Finally, two of these plasmids were sequenced, one with the forward and the second with the reverse primer so as to detect potential PCR mutations.

1.5 Identification of IS Element Insertion Site

PCR primer pairs, used to confirm the DNA integrity and sequence, were selected from around the IS element and used to verify the chromosomal DNA environment of other isolates of S. macedonicus from the Nestlé Culture Collection. The first primer pair has one oligonucleotide, 9411 (^{5′}ACAGGTACCTTGTCTGGAAATGCAGAG³′) (SEQ ID NO:11), within the epsD gene 5′ to the IS element and the second, 9412 (^{5′}CTCGGATCCAACCGCTCTATCTGCTGC^{3′}) (SEQ ID NO:12), within the IS element. Similarly, the second primer pair has one oligonucleotide, 9413 (^{5′}TCCGGTACCTTTCTCTTGTAGTGACCG^{3′}) (SEQ ID NO:13), within the IS element and the second, 9414 (^{5 ′}CGTGGATCCCGTGACAAACACTACCTG^{3′}) (SEQ ID NO:14), within the epsE gene positioned 3′ to the IS element. One of the strains tested, CNCM I-1926, showed no PCR amplification product with either of the primer pairs 9411+9412 and 9413+9414, but produced a smaller than predicted amplification product with the primer pair 9411+9414, flanking the IS element. The size of this PCR product corresponded to the predicted size of this genomic region without the IS element. This PCR product was cloned, its DNA sequence determined and compared to that of CNCM I-1923.

1.6 Bioinformatic Analysis

The DNA sequence was compiled using the GelAssemble program from the GCG suite of programs (Wisconsin Package Version 10.0, Genetics Computer Group (GCG), Madison, Wis. USA). The consensus sequence was exported to the GeneWorks program for prediction of open reading frames, which were compared against the bacterial DNA sequence subset of GenBank (release 110.0) and EMBL (release 58.0) databases using the tFasta program in the GCG suite. Finally, each potential protein hit was extracted and compared to the S. macedonicus protein using the Bestfit program from GCG.

2 Results and Discussion

2.1 Identification of the CNCM I-1923 Exopolysaccharide Production Operon

2.1.1 Identification of an EpsA homologue in CNCM I-1923

The DNA sequence of the EPS operon of from Sfi6 was used to design PCR primer pairs to amplify the epsA, epsJ, eps L and epsM genes from CNCM I-1923 chromosomal DNA. From these PCR reactions, only the epsA primer pairs produced an amplification product whose approximate size of 1200 base-pairs corresponds well with that predicted from the Sfi6 epsA gene (SEQ ID NO:16). This PCR product was cloned, its DNA sequence determined and compared to that of the Sfi6 epsA gene (SEQ ID NO:16). The result of the bioinformatic analysis is shown in FIG. 3, where a very significant 96.8% DNA sequence identity was revealed, indicating that this is a homologue of the Sfi6 epsA gene (SEQ ID NO:16). This analysis also identifies a ten base-pair deletion at position 834 within the CNCM I-1923 epsA gene and results in the premature termination of the protein seven amino acids later.

While the regulatory genes of many polysaccharide synthesis operons show a significant level of similarity, the role of this DNA sequence in polysaccharide production in CNCM I-1923 could only be proven by cloning and sequencing adjacent genes. To this end, more template DNA surrounding the epsA gene was cloned or generated by inverted PCR and its DNA sequence determined. Bioinformatic analysis confirmed the presence of genes involved in EPS production surrounding the epsA gene.

The DNA sequence, open reading frame prediction and analysis will be presented in greater detail later, but initial analysis had revealed the presence of an IS element, two epsA genes and one complete and one truncated epsB homologues. The arrangement of the genes was confirmed by the PCR amplification of short overlapping segments directly from the CNCM I-1923 chromosomal DNA. These amplified fragments were also cloned to confirm the remaining ambiguities present in the DNA sequence to produce a publication and patent quality sequence.

2.1.2 DNA Sequencing of the entire CNCM I-1923 Exopolysaccharide Operon

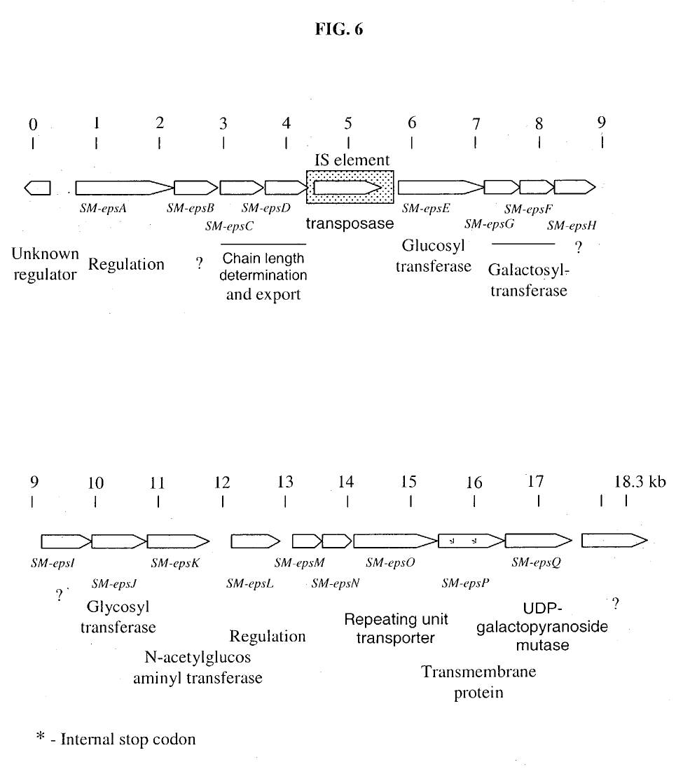

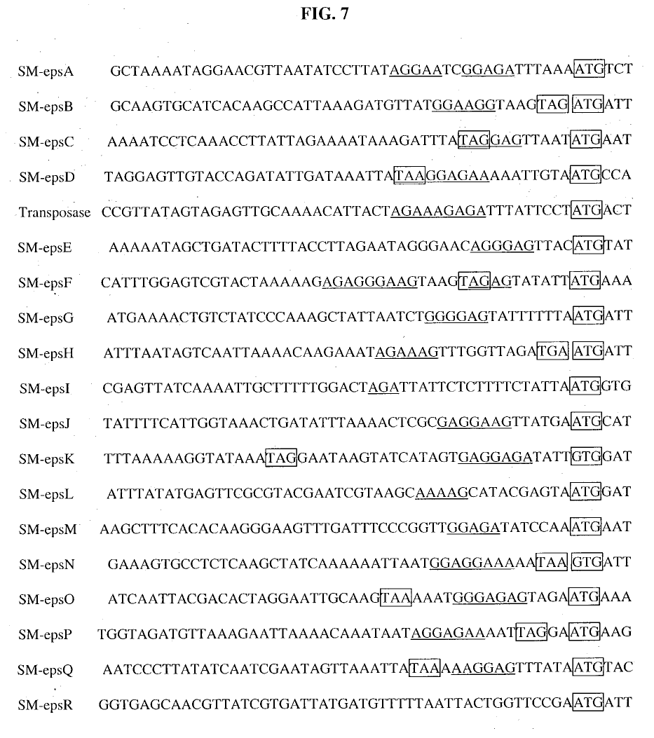

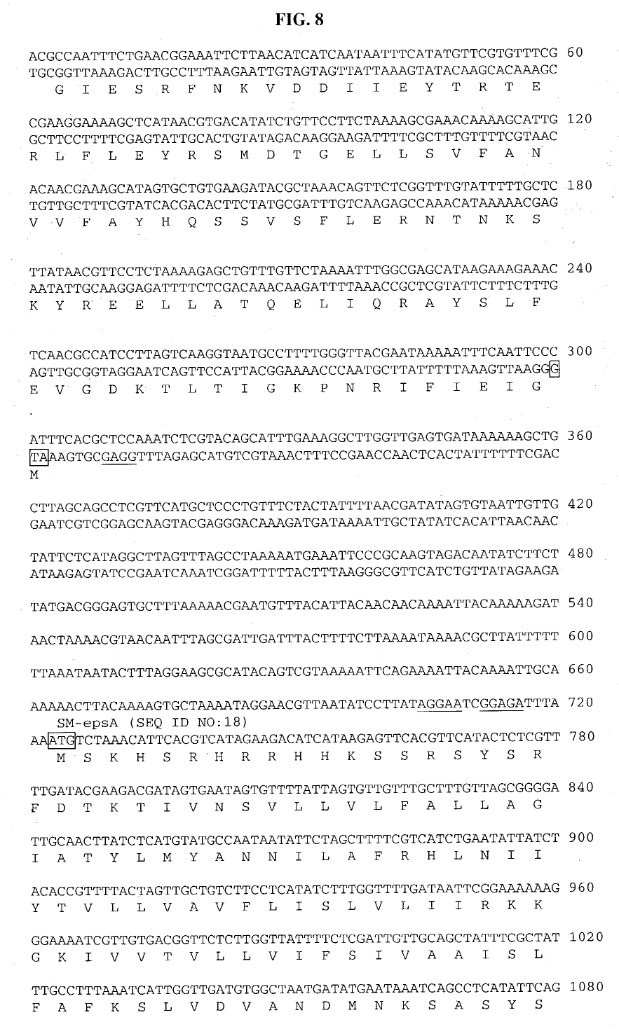

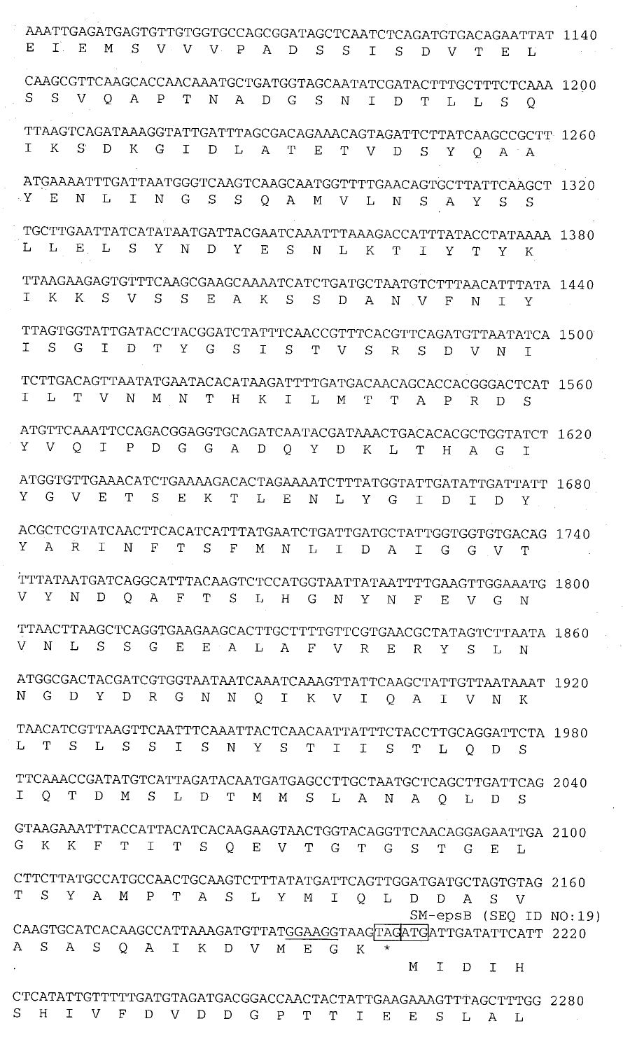

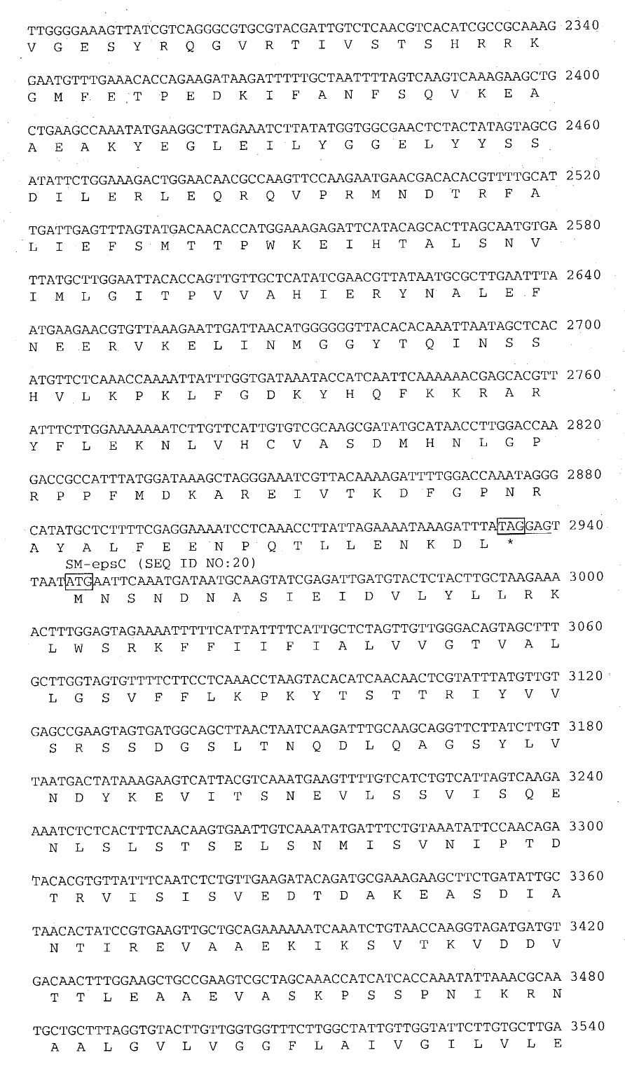

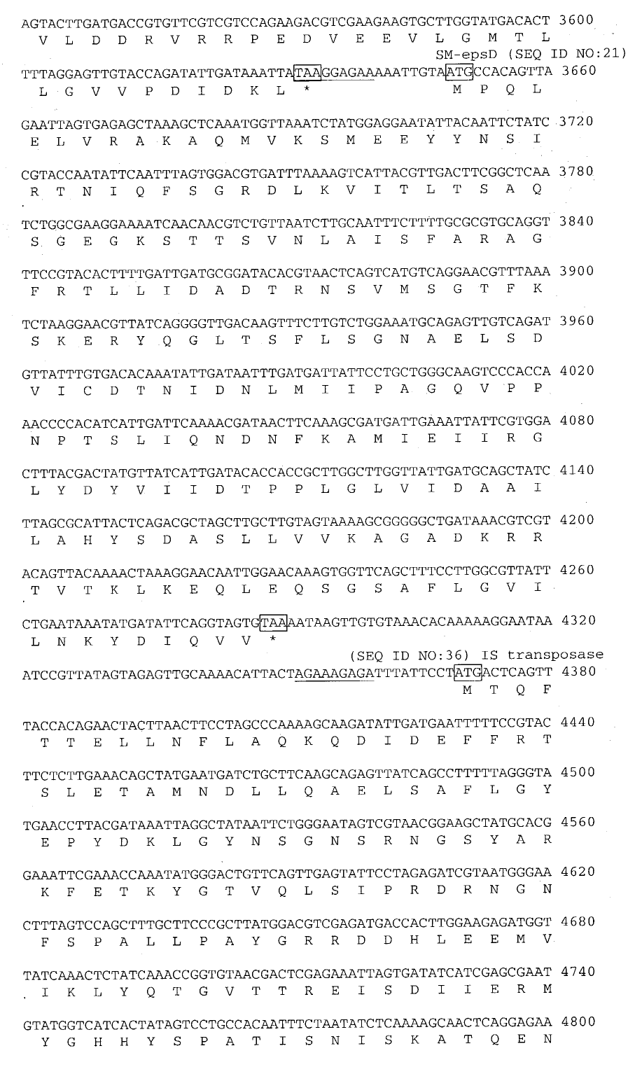

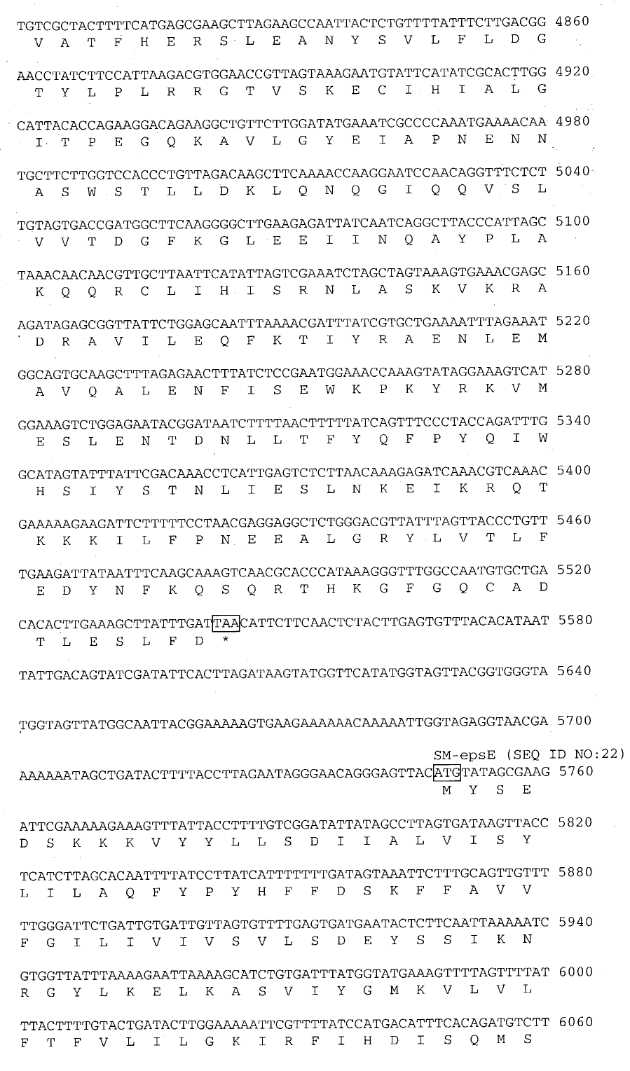

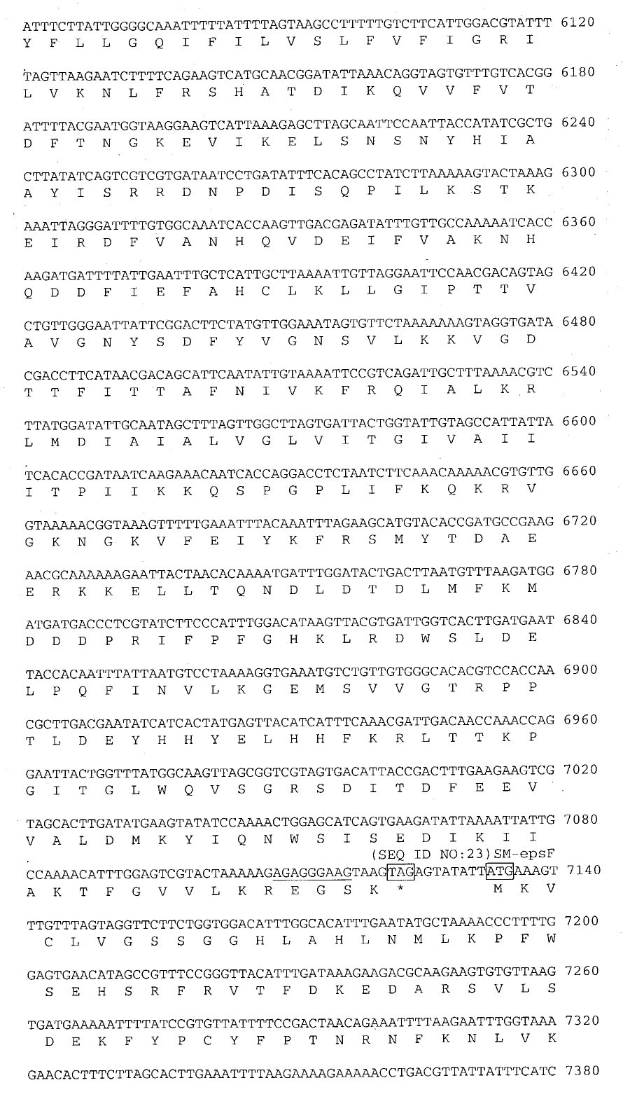

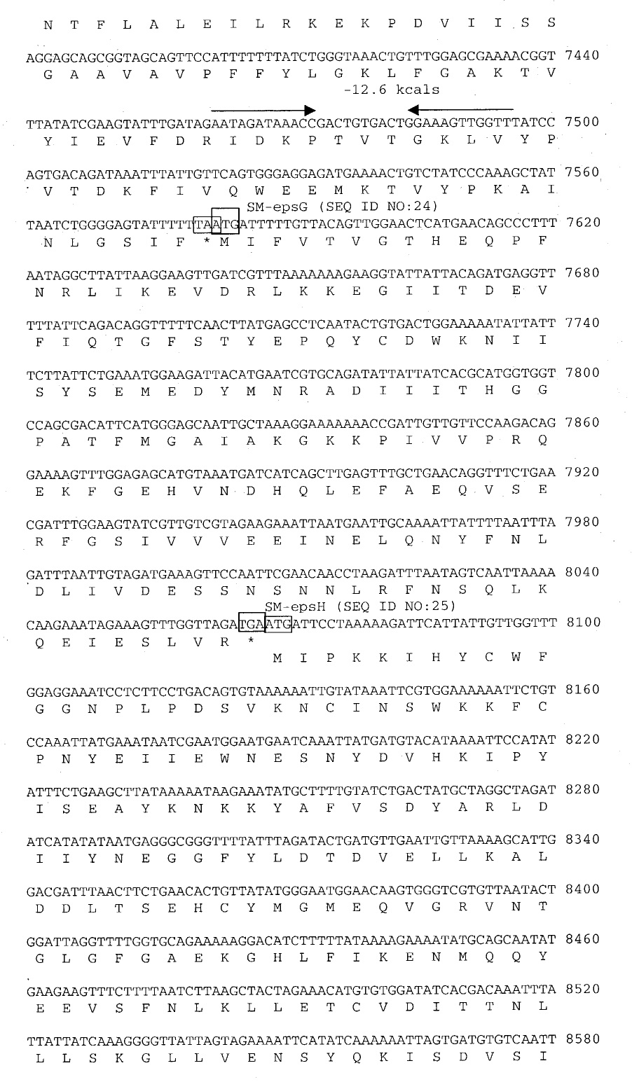

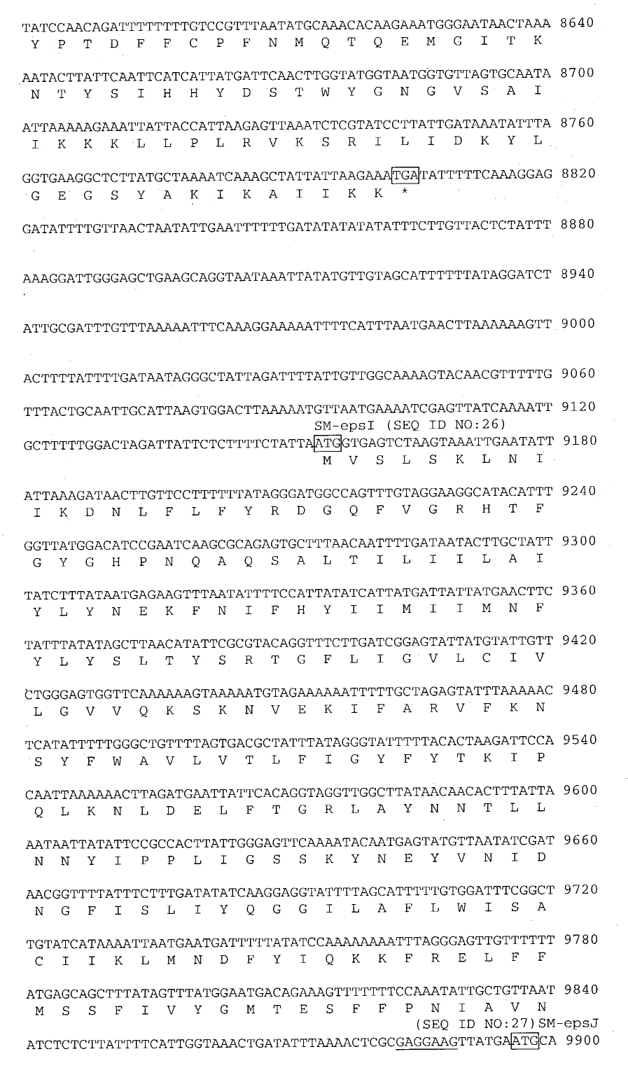

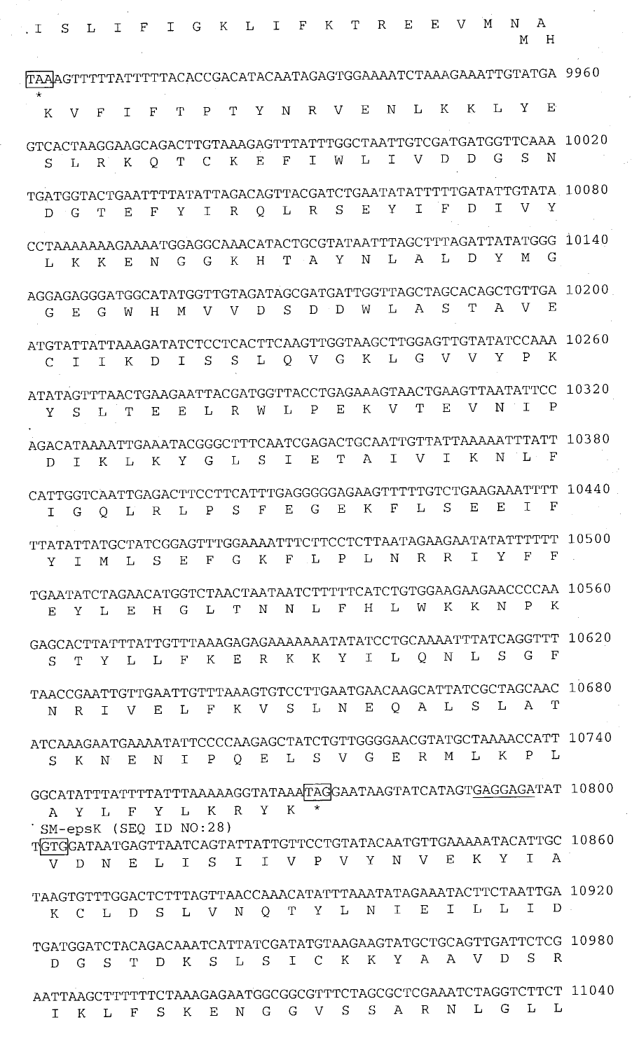

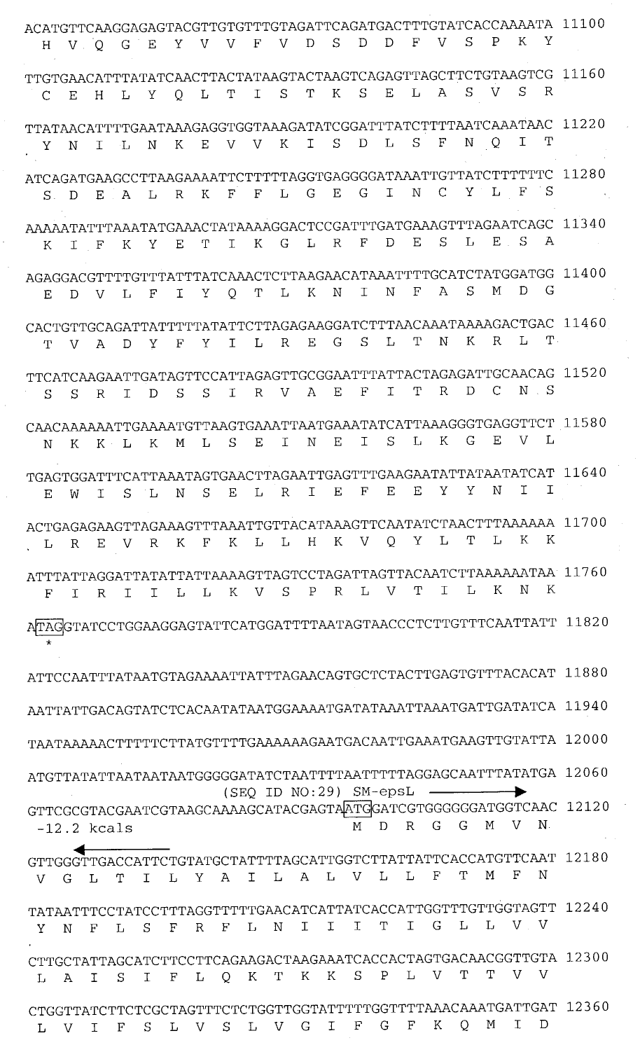

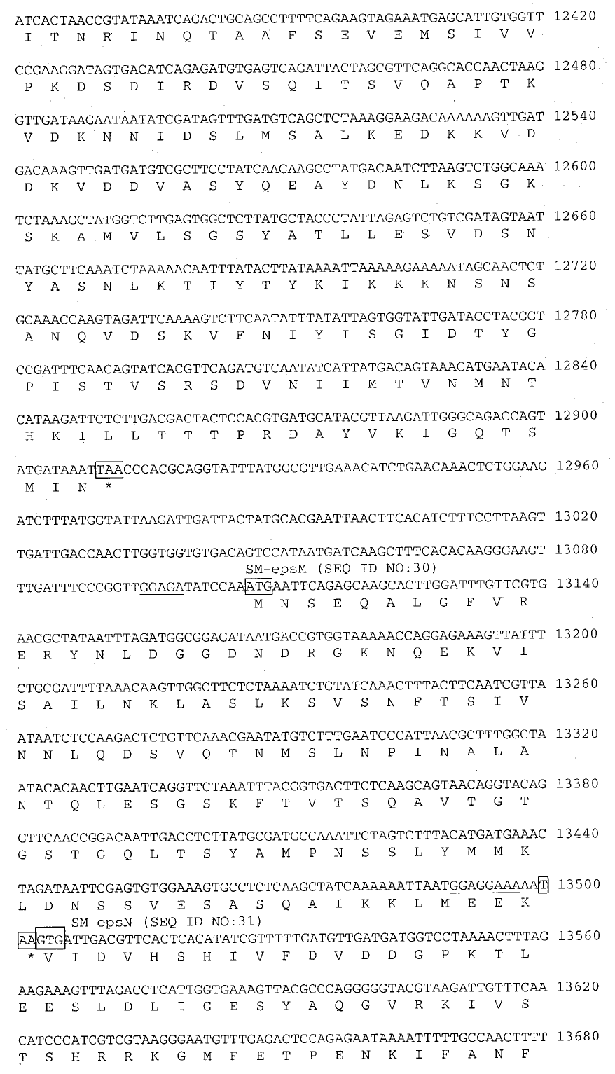

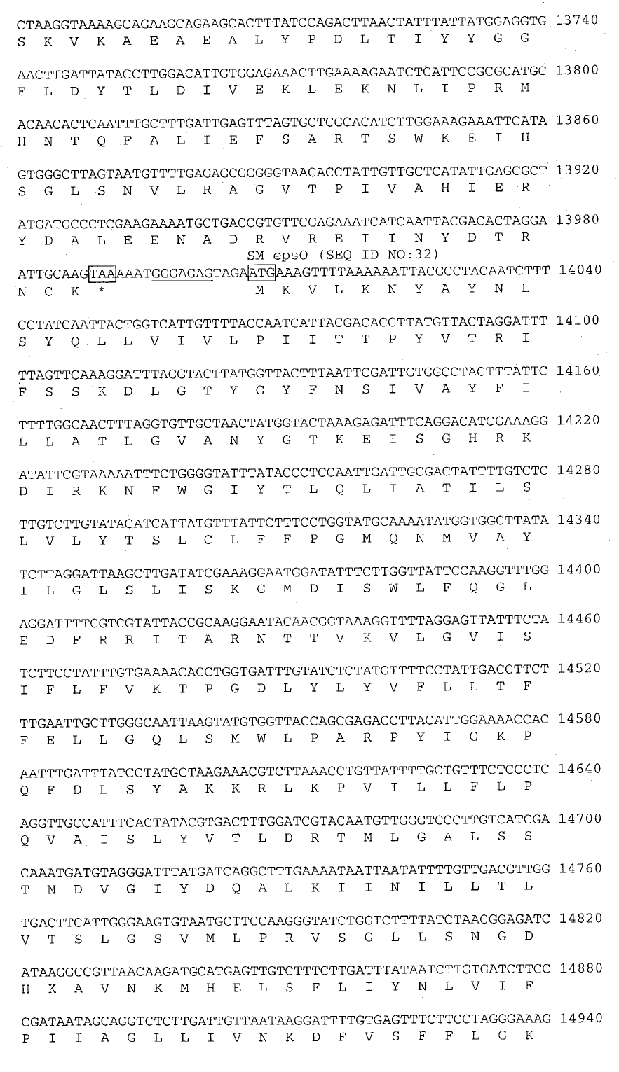

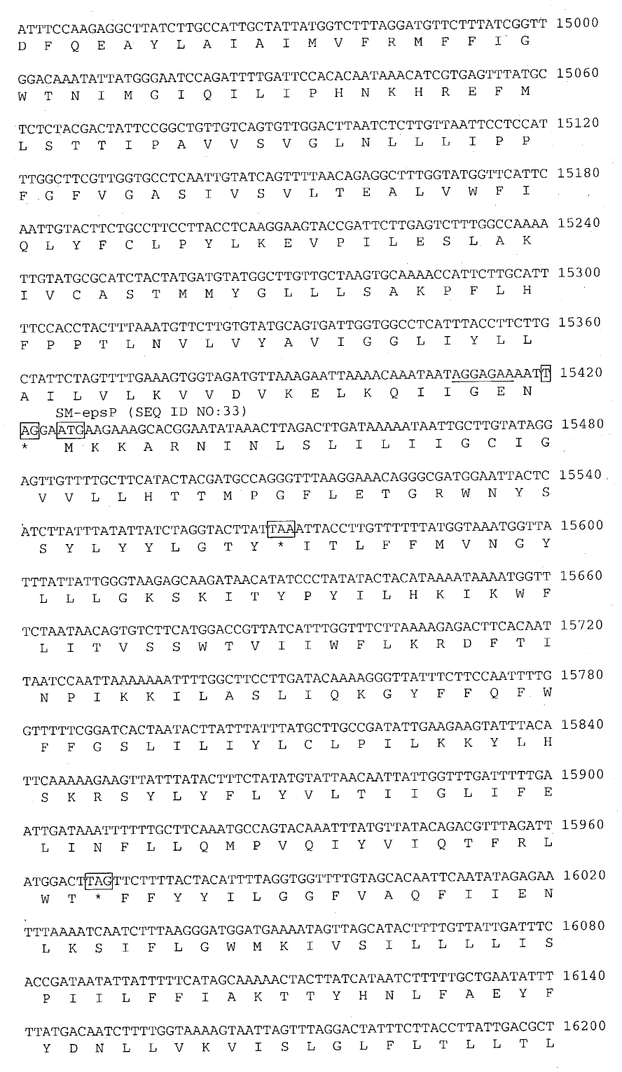

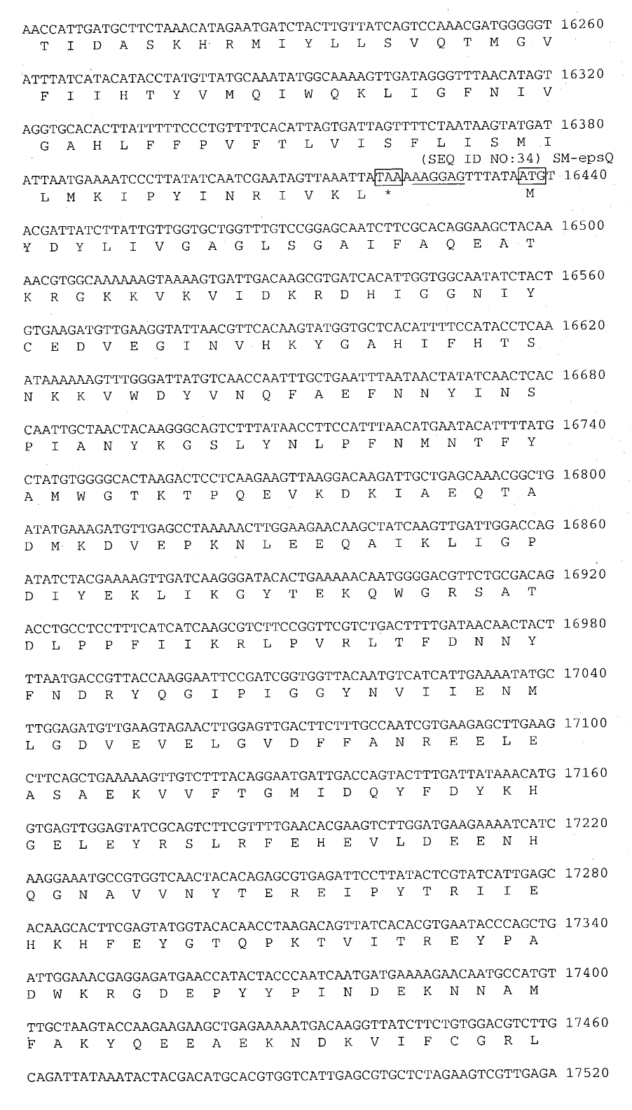

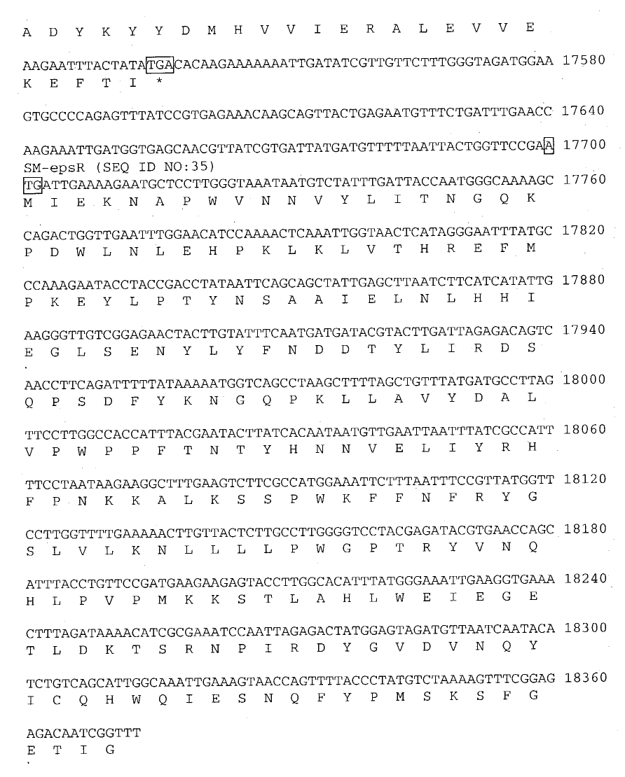

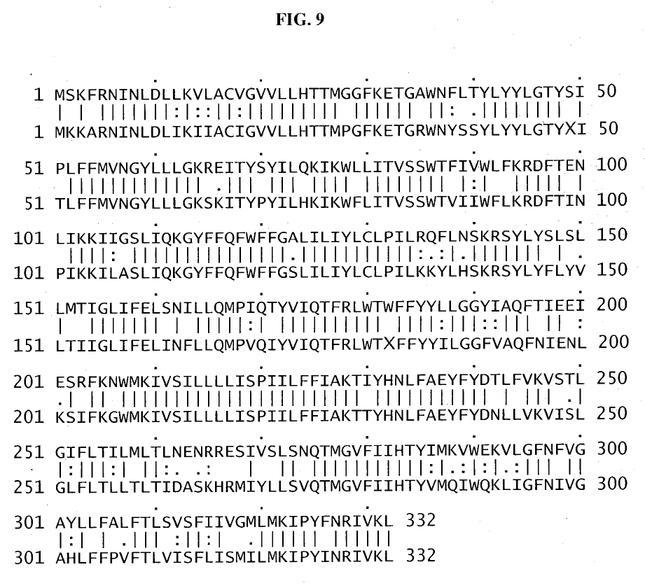

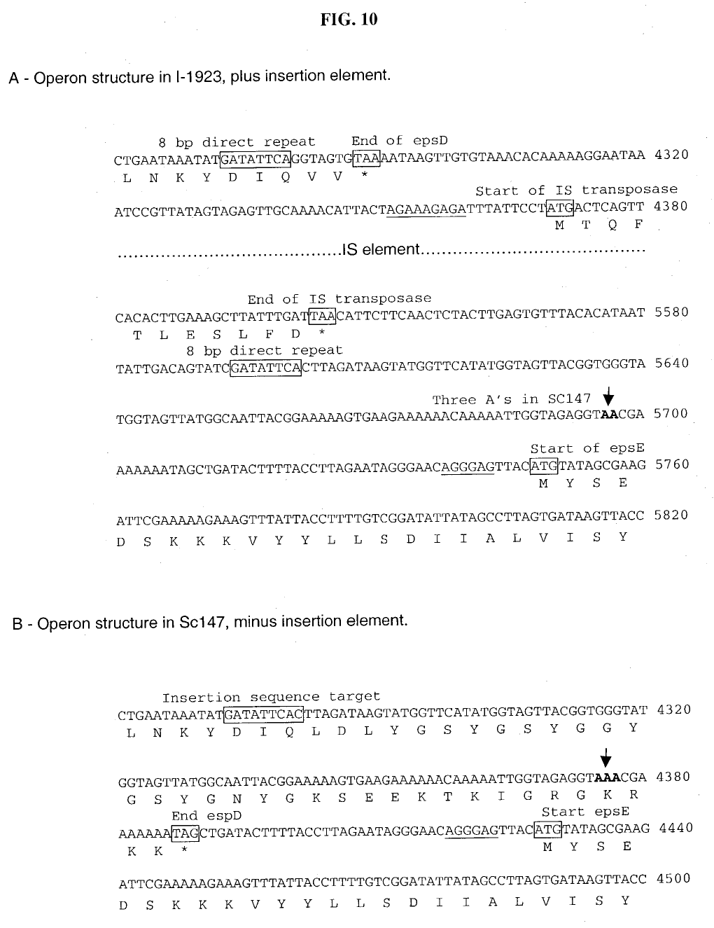

The present invention provides a DNA sequence (SEQ ID NO:4) of 18,372 base pairs of the S. macedonicus operon for the production of exocellular polysaccharide. This information was derived from a single cloned DNA fragment supplemented by inverted PCR products. A map of the operon with its predicted open reading frames is shown in FIG. 6, while the DNA sequence plus protein translation products are shown in FIG. 8.

2.2 Analysis of the CNCM I-1923 EPS Operon

2.2.1 General Structure

Of the 21 predicted open reading frames, 15 show clear similarities to proteins from previously identified exopolysaccharide synthesis operons from food or pathogenic bacteria. These results are presented in Table 1, while Table 2 contains physical information of the predicted proteins and their predicted function, inferred by similarity. FIG. 7 aligns the proposed initiation codons from the predicted translation products and also indicates the most probable ribosome binding site, the DNA sequence motif to which the bacterial ribosomal complex attaches as a pre-requisite to translation of the mRNA into protein.

From the remaining predicted translation products, three lie within the eps operon. One of these appears to be part of an insertion element, while the remaining two are of no known function. From the flanking predicted translation products, the gene positioned at the start of the eps operon is translated in the opposite direction and encodes the start of a probable regulator for an unknown operon. The second flanking predicted gene, positioned at the end of the eps operon, shows no significant similarity to any proteins or genes at present in the databases.

Analysis of the DNA sequence with the GCG program Stemloop to find the presence of probable terminator structures revealed only a such few structures with a reasonably strong hybridization energies.

The overall content of G+C nucleotides in this operon is relatively low, at approximately 34%.

TABLE 1
Listing of the predicted proteins from the S. macedonicus eps operon with the Bestfit
scores for identity and, in brackets, similarity.

| | SM- | SM- | SM- | SM- | SM- | SM- | SM- | SM- | SM- |
|---|---|---|---|---|---|---|---|---|---|
| | epsA | epsB | epsC | epsD | epsE | epsF | epsG | epsH | epsI |
| S. | epsA | epsB | epsC | epsD | — | — | — | — | — |
| therm | 61.4 | 73.3 | 61.6 | 65.6 | | | | | |
| Sfi6 | (70.7) | (79.0) | (68.6) | (73.1) | | | | | |
| S. sali | cpsA | cpsB | cpsC | cpsD | cpsE | End of | — | — | — |
| cps | 70.0 | 72.8 | 61.6 | 70.0 | 41.1 | seq | | | |
| (1) | (75.4) | (78.6) | (68.1) | (74.0) | (53.2) | | | | |
| S. pneu | cpsA | cpsB | cpsC | cpsD | cpsE | cpsF | cpsG | — | — |
| cps 14 | 55.0 | 64.3 | 55.0 | 59.0 | 37.0 | 81.9 | 55.7 | | |
| (2) | (63.6) | (75.6) | (65.3) | (70.8) | (49.8) | (88.6) | (65.8) | | |
| S. pneu | cps19f | cps19f | cps19f | cps19f | cps19f | No sim | — | — | — |
| cps | A | B | C | D | E | | | | |
| 19F | 53.8 | 66.0 | 53.6 | 59.0 | 36.7 | | | | |
| (3) | (62.8) | (77.3) | (66.2) | (72.2) | (49.7) | | | | |
| S. pneu | cap33f | cap33f | cap33f | cap33f | cap33f | — | — | — | — |
| cps | A | B | C | D | E | | | | |
| 33F | 55.1 | 64.7 | 53.6 | 56.6 | 37.3 | | | | |
| (4) | (63.3) | (76.0) | (63.5) | (68.4) | (50.7) | | | | |
| S. alag | (incom | cpsA | cpsB | cpsC | cpsD | cpsE | cpsF | — | — |
| eps | plete) | 72.9 | 50.7 | 60.0 | 41.0 | 82.6 | 55.8 | | |
| (5) | 55.6 | (80.8) | (62.9) | (72.6) | (52.0) | (87.9) | (64.3) | | |
| | (61.9) | | | | | | | | |
| L. | — | epsC | — | epsB | — | epsE | epsF | — | — |
| lactis | | 27.8 | | 40.6 | | 37.0 | 43.0 | | |
| eps | | (38.9) | | (50.0) | | (50.0) | (53.1) | | |
| (6) | | | | | | | | | |
| St. | — | capC | capA | capB | — | — | | — | — |
| aureus | | 34.1 | 29.5 | 35.6 | | | | | |
| M eps | | (47.8) | (42.4) | (49.0) | | | | | |
| (7) | | | | | | | | | |

| | SM-eps | SM-eps | SM-eps | SM-eps | SM-eps | SM-eps | SM-eps | SM-eps |
|---|---|---|---|---|---|---|---|---|
| | J | K | L | M | N | O | P | Q |
| S. | epsI | | epsA | epsA | epsB | — | — | — |
| therm | 33.3 | | 95.8 | 98.5 | 96.1 | | | |
| Sfi6 | (44.2) | | (97.7) | (98.5) | (96.1) | | | |
| S. sali | — | — | No sim | cpsA | cpsB | — | — | — |
| cps | | | | 93.8 | 98.1 | | | |
| | | | | (94.6) | (98.1) | | | |
| S. pneu | — | cpsJ | cpsA | cpsA | cpsB | cpsL | — | — |
| cps 14 | | 38.1 | 47.5 | 56.3 | 63.2 | 34.7 | | |
| | | (53.2) | (56.8) | (62.5) | (72.3) | (47.4) | — | — |
| S. pneu | — | — | cps19fA | cps19fA | cps19tB | | | |
| cps 19F | | | 47.1 | 54.7 | 63.9 | | | |
| | | | (56.0) | (61.0) | (72.9) | | | |
| S. pneu | cap33fH | cap33fJ | — | — | — | cap33fL | Cap33f | cap33fN |
| cps 33F | 29.5 | 32.5 | | | | 77.9 | M | 95.3 |
| | (40.4) | (44.9) | | | | (84.7) | 73.8 | (97.5) |
| | | | | | | (81.3) | | |
| S. alag | cpsH | cpsH | No sim | cpsX | cpsA | — | — | — |
| cps | 36.9 | 34.9 | | 64.6 | 70.3 | | | |
| | (45.6) | (46.9) | | (69.3) | (75.5) | | | |
| L. lactis | epsG | — | No sim | No sim | No sim | epsK | — | — |
| Eps | 30.2 | | | | | 33.6 | | |
| | (42.5) | | | | | (46.5) | | |
| St. | ?— | — | — | — | — | — | — | — |
| aureus | | | | | | | | |
| M cps | | | | | | | | |

| | |
|---|---|
| (1) Streptococcus salivarius: | GeneBank Accession X94980 |
| (2) Streptococcus pneumoniae cps14: | GeneBank Accession X85787 |
| (3) Streptococcus pneumoniae cps19F: | GeneBank Accession SPU09239 |
| (4) Streptococcus pneumoniae cps33F: | GeneBank Accession AJ006986 |
| (5) Streptococcus agalactiae strain COH1: | GeneBank Accession AB017355 |
| (6) Lactococcus lactis plasmid pNZ4000: | GeneBank Accession LLU93364 |
| (7) Staphalococcus aureus M type 1: | GeneBank Accession SAU10927 |

(1) Griffin A. M., Morris V. J. and Gasson M. J. (1996) The cpsABCDE genes involved in polysaccharide production in Streptococcus salivarius ssp. thermophilus strain NCBF 2393. Gene 183:23-27.

(2) Kolkman M. A., Wakarchuk W., Nuijten P. J. and van der Zeijst B. A. (1997) Capsular polysaccharide synthesis in Streptococcus pneumoniae serotype 14: molecular analysis of the complete cps locus and identification of genes encoding glycosyltransferases required for the biosynthesis of the tetrasaccharide subunit. Mol. Microbiol. 26:187-208.

(3) Morona J. K., Morona R. and Paton J. C. (1997) Characterization of the locus encoding the Streptococcus pneumoniae type 19F capsular polysaccharide biosynthesis pathway. Mol. Microbiol. 23:751-763.

(4) Llull D., Lopez R., Garcia E. and Munoz R. Data submitted to GenBank, but not found as published.

(5) Yamamoto S., Miyake K. and Iijima S. Data submitted to GenBank, but not found as published.

(6) van Kranenburg R., Marugg J. D., van Swam I. I. Willem N. J. and de Vos W. M. (1997) Molecular characterization of the plasmid-encoding eps gene cluster essential for exopolysaccharide biosynthesis in Lactococcus lactis. Mol. Microbiol. 24:387-397.

(7) Lin W. S., Cunneen T. and Lee C. Y. (1994) Sequence analysis and molecular characterization of genes required for the biosynthesis of type 1 capsular polysaccharide in Staphylococcus aureus. J. Bacteriol. 176:7005-7016.

TABLE 2
Listing of the S. macedonicus exopolysaccharide operon protein
information and the probable functions.

| | Length | Mass | |
|---|---|---|---|
| Protein | (aa) | (da) | Probable function |
| SM-epsA | 493 | 53850 | BPS operon regulator. |
| SM-epsB | 243 | 28041 | Unknown function in EPS operon. |
| SM-epsC | 229 | 24884 | EPS export. |
| SM-epsD | 213 | 23312 | EPS export. |
| SM-epsE | 450 | 52550 | Putative glucosyl-1-phosphate transferase. |
| SM-epsF | 149 | 17043 | Putative galactosyltransferase. |
| SM-epsG | 161 | 18598 | Putative galactosyltransferase. |
| SM-epsH | 245 | 28286 | No similarities. |
| SM-epsI | 249 | 28972 | No similarities. |
| SM-epsJ | 292 | 34362 | Putative glycosyltransferase. |
| SM-epsK | 320 | 36856 | Putative N-acetylglucosaminyltransferase. |
| SM-epsL | — | — | Partial, EPS operon regulator. |
| SM-epsM | — | — | Partial, EPS operon regulator. |
| SM-epsN | — | — | Partial, unknown function in EPS operon. |
| SM-epsO | 471 | 52839 | Repeating unit transporter. |
| SM-epsP | — | — | Transmembrane protein. (Three peptides) |
| SM-epsQ | 366 | 42717 | UDP-galactopyranoside mutase. |

5 2.2.1.1 SM-epsA (SEQ ID NO:18)

The first gene in the eps operon of S. macedonicus, SM-epsA, is preceded by a good ribosome-binding site and encodes a predicted protein of 493 amino acids and a mass of 53.85 kDa. The SM-epsA predicted protein shows similarities to many predicted regulation proteins from eps and cps operons. These proteins possess a potential ‘helix-turn-helix’ DNA-binding motif in their N-terminal section and are transcription activators that usually negatively regulate their own expression. The present invention provides that the SM-epsA gene is the regulator of the eps operon.

2.2.1.2 SM-epsB (SEQ ID NO:19)

The second gene in the operon, SM-epsB, is preceded by a good ribosome-binding site and encodes a predicted protein of 243 amino acids (28.04 kDa). SM-epsB shows strong similarities to many homologous proteins encoded by eps and cps operons (that occupy the same position in the operon), but to date, no function has yet been assigned to the protein.

2.2.1.3 SM-epsC (SEQ ID NO:20)

The third gene in the operon, SM-epsC, is preceded by a good ribosome-binding site and encodes a predicted protein of 229 amino acids (24.88 kDa). The SM-epsC protein shows a strong homology to other eps/cps proteins, most of which occupy a similar third position in the operon. By sequence similarity, these proteins, together with the following protein, SM-epsD, are involved in the regulation of the exopolysaccharide chain length.

2.2.1.4 SM-epsD (SEQ ID NO:21)

The fourth gene in the operon, SM-epsD, is preceded by a good ribosome-binding site and encodes a predicted protein of 213 amino acids (23.31 kDa). The SM-epsD protein contains a so-called P-loop motif required for ATP/GTP binding and could be part of the ABC-transporter apparatus. This is consistent with the role of SM-epsD in chain length determination and transport of the repeating units. Finally, the bioinformatic analysis shows that the SM-epsD protein is truncated in relation to the related cps proteins, which could indicate that the IS element has inserted within the SM-epsD gene, close to the carboxy-terminus. This will be discussed later in relation to the IS element.

2.2.1.5 SM-epsE (SEQ ID NO:22)

The sixth gene in the operon, SM-epsE, is preceded by a good ribosome-binding site and encodes a predicted protein of 450 amino acids (52.55 kDa). The SM-epsE protein shows strong similarities (approximately 40% identity) to five glucosyl-1-phosphate transferases from the exopolysaccharide synthesis operons of the genus Streptococcus.

2.2.1.6 SM-epsF (SEQ ID NO:23)

The seventh gene in the operon, SM-epsF, is preceded by a good ribosome-binding site and encodes a predicted protein of 149 amino acids (17.04 kDa). The SM-epsF protein shows strong similarities (approximately 80% protein identity) to the S. pneumoniae serotype 14 cpsF and S. alagactiae cpsF proteins. See below for predicted function.

2.2.1.7 SM-epsG (SEQ ID NO:24)

The eighth gene in the operon, SM-epsG, is preceded by a good ribosome-binding site and encodes a predicted protein of 161 amino acids (18.60 kDa). The SM-epsG protein shows three strong sequence similarities to the S. pneumoniae serotype 14 cpsG, S. alagactiae cpsF and L. lactis epsF proteins (between 43.0 to 55.0% sequence identity). Experimental results obtained with S. pneumoniae serotype 14 show that the 14 cpsF and 14 cpsG proteins associate to form an active galactosyltransferase. The present invention provides that SM-epsF and SM-epsG together encode a galactosyltransferase.

2.2.1.8 SM-epsH (SEQ ID NO:25)

The ninth gene in the operon, SM-epsH, is preceded by a good ribosome-binding site and encodes a predicted protein of 245 amino acids (28.29 kDa). The SM-epsH protein does not show any significant similarities to any translated bacterial DNA sequence in the GenBank data bank.

2.2.1.9 SM-epsI (SEQ ID NO:26)

The tenth gene in the operon, SM-epsI, is preceded by a weak ribosome-binding site and encodes a predicted protein of 249 amino acids (28.97 kDa). The SM-epsI protein does not show any significant similarities to any translated bacterial DNA sequence in the GenBank or EMBL data banks. The SM-epsI protein contains a sequence motif required for lipoprotein synthesis. In prokaryotes, these proteins are synthesized with a precursor signal peptide, which is cleaved by a specific lipoprotein signal peptidase (signal peptidase II). The peptidase recognizes a conserved sequence and cuts upstream of a cystein residue to which a glyceride-fatty acid lipid is attached (Hayashi S. and Wu H. C. (1990) Lipoproteins in bacteria. J. Bioenerg. Biomembr. 22(3): 451-71). Hence SM-epsI is predicted to be a membrane-associated protein.

2.2.1.10 SM-epsJ (SEQ ID NO:27)

The eleventh gene in the operon, SM-epsJ, is preceded by a good ribosome-binding site and encodes a predicted protein of 292 amino acids (34.36 kDa). The SM-epsJ protein shows some extended, but low sequence similarity to the amino terminal 200 amino acids of the S. pneumoniae serotype 33F cap33fH protein. This protein is described as a glycosyltransferase.

2.2.1.11 SM-epsK (SEQ ID NO:28)

The twelfth gene in the operon, SM-epsK, is preceded by a good ribosome-binding site and encodes a predicted protein of 320 amino acids (36.86 kDa). The SM-epsK protein shows a good sequence similarity over the complete length of the S. agalactiae cpsH protein, described as an N-acetylglucosaminyltransferase.

2.2.1.12 SM-epsL (SEQ ID NO:29) and SM-epsM (SEQ ID NO:30)

The thirteenth and fourteenth genes in the operon, SM-epsL and SM-epsM, encode predicted proteins with a very high level of similarity to the S. thermophilus Sfi6 epsA protein, the predicted regulator of the eps operon. It was this gene that was originally isolated using PCR primers derived from the S. thermophilus Sfi6 operon, and as can be seen in FIG. 3, contains a ten base-pair deletion in the SM-epsA gene relative to the Sfi6 epsA gene which accounts for the frame shift and the presence of two partial proteins. Taken with the fact the both genes are not preceded by recognizable ribosome-binding sites, it is sure that this gene does not produce an active regulator for the S. macedonicus eps operon.

2.2.1.13 SM-epsN (SEQ ID NO:31)

The fifteenth gene the operon, SM-epsN, encodes a predicted protein of approximately 160 amino acids with a very strong similarity (96% identity) to the first 160 amino acids (out of 243 amino acids) of the S. thermophilus Sfi6 epsB protein. The SM-epsN gene translation initiation codon (GTG) is preceded by a good ribosome-binding site. It is predicated that this epsB homologue is also no longer active.

2.2.1.14 SM-epsO (SEQ ID NO:32)

The sixteenth gene in the operon, SM-epsO, is preceded by a good ribosome-binding site and encodes a predicted protein of 471 amino acids (52.84 kDa). The SM-epsO protein shows a strong similarity to the repeating unit transporter protein from the S. pneumoniae serotype 33F cps33 fL protein and is most probably involved in the export of the repeating unit.

2.2.1.15 SM-epsP (SEQ ID NO:33)

The seventeenth gene in the operon, SM-epsP, is preceded by a strong ribosome-binding site and encodes a potential protein with a strong, 73.8% identity to the transmembrane protein cap33fM, of the S. pneumoniae 33F cps operon. The SM-epsP protein also contains the P-loop motif required for ATP/GTP binding. While this protein would normally be expected to be involved in the transport of the repeating unit, the SM-epsP gene contains two internal translation termination codons that effectively truncates the protein at positions 49 and 182. Bioinformatic analysis reveals that the similarity of the SM-epsP protein to cap33fM is continuous through-out the length, but is broken into three separate parts by the presence of the two stop codons as can be seen in FIG. 7. While this situation has been seen in other eps operons, its significance is not yet understood.

2.2.1.16 SM-epsQ (SEQ ID NO:34)

The eighteenth and last gene in the operon, SM-epsQ, is preceded by a good ribosome-binding site and encodes a predicted protein of 366 amino acids (42.72 kDa). This protein shows a strong similarity to the S. pneumoniae serotype 33F cap33fN protein, a predicted UDP-galactopyranose mutase. This enzyme is involved in sugar conversion in lipopolysaccharide biosynthesis where it catalyses the conversion of UDP-D-galactopyranose into UDP-D-galacto-1,4-furanose (Nassau P. M., Martin S. L., Brown R. E., Weston A., Monsey D., McNeil M. R. and Duncan K. (1996) Galactofuranose biosynthesis in Escherichia coli K-12: identification and cloning of UDP-galactopyranose mutase. J. Bacteriol. 178:1047-1052).

2.2.1.17 Flanking Regions

The two genes flanking the above described S. macedonicus exopolysaccharide genes show no similarity to any previously described cps or eps genes. The open-reading frame to the 5′ of the S. macedonicus eps operon is transcribed in the opposite direction to the eps operon and contains a potential ‘helix-turn-helix’ DNA-binding motif in its N-terminal section and is hence probably a transcription activator of the adjacent, unrelated operon. The open-reading frame 3′ to the eps operon shows no significant similarities to any translated bacterial DNA sequence in the GenBank or EMBL data banks.

2.2.2 Repeating Unit Synthesis

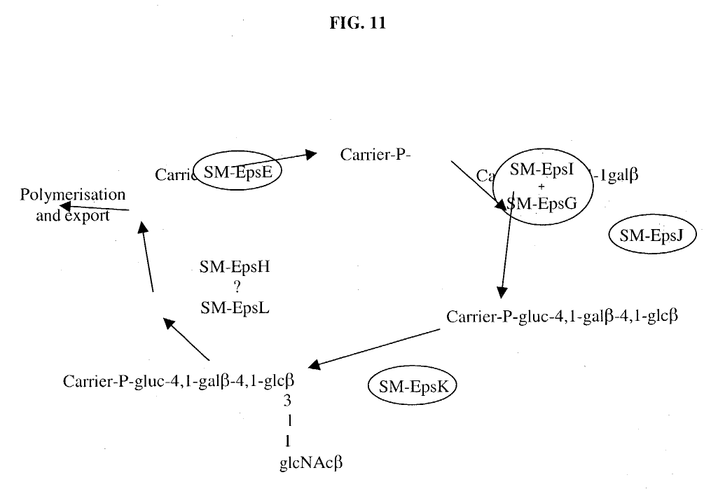

From these gene/protein designations, the present invention provides a pathway for the synthesis of the oligosaccharide repeating unit and associate this with the predicted enzymatic activities. The addition of each sugar unit requires a unique sugar transferase. The two unidentified genes, SM-epsH (SEQ ID NO:25) and SM-epsI, (SEQ ID NO:29) most probably encode these missing functions. A prediction of the oligosaccharide repeating unit synthesis pathway has been constructed and is shown in FIG. 11.

2.2.3 The IS Element of CNCM I-1923 (SEQ ID NO:35)

FastA analysis of the predicted open reading frames from the EPS operon of CNCM I-1923 identified one gene with a very high protein identity to the transposase from the S. thermophilus insertion sequence IS 1191 (Guedon G., Bourgoin F., Pebay M., Roussel Y., Colmin C., Simonet J. M. and Decaris B. (1995) Characterization and distribution of two insertion sequences, IS 1191 and iso-IS981, in Streptococcus thermophilus: does intergeneric transfer of insertion sequences occur in lactic acid bacteria co-cultures?. Mol. Microbiol. 16:(1), 69-78). BestFit pairwise comparison of the translated protein sequences revealed a very high 97.95% identity and a 98.21% similarity over the complete length of the proteins. This high level of similarity between the CNCM I-1923 IS element and IS1191 from S. thermophilus is also seen at the DNA sequence level, with a 99.01% identity over 1313 bp and corresponds exactly to the published size of IS1191. IS1191 and our S. macedonicus IS element has 28 bp imperfect terminal inverted repeats and both elements potential encode a single protein of 391 amino acids, the probable transposase. This high sequence identity between the two IS elements is a strong evidence for a recent lateral gene transfer between the two species. The IS1191-like element in S. macedonicus has inserted into the end of the epsD gene, possibly prematurely terminating this protein.

Screening of the remaining S. macedonicus strains in the Nestlé Culture Collection identified CNCM I-1926 as lacking this IS1191-like element described in strain CNCM I-1923. The DNA sequence of this region was determined from CNCM I-1926 and confirms the presence an 8 bp target duplication upon insertion of the element (again, in agreement with IS1191). Additionally, the insertion of the element has caused the pre-mature termination of the epsD gene, eliminating a predicted 45 amino acids from the carboxy-terminus. While this protein is important for the exopolysaccharide biosynthesis, its truncation has not adversely affected the synthesis in strain CNCM I-1923, which was targeted as the highest (marginally) exopolysaccharide producer.

3 Conclusions

The present invention provides a DNA sequence and bioinformatic analysis of the exopolysaccharide production operon from the food-grade lactic acid bacterium S. macedonicus. This bacterium produces a branched polysaccharide with a composition close to that of human maternal milk and could be interesting to include in infant formulae or in some medical applications.

The S. macedonicus exopolysaccharide operon encodes for proteins with strongest similarities to both food-grade and pathogenic streptococci, with only very limited similarity to the operons of Lactobacillus bulgaricus or L. helveticus (data provided by the Glycobiology Group and analysis not reported here). The exopolysaccharide operon of S. macedonicus strain CNCM I-1923 contains identified elements for almost all the required functions, including regulation, transferases for the addition of the sugars, a transport and chain length determination system. The operon shows much evidence of lateral gene transfer from streptococci. The most striking evidence is the presence of the insertion element which shows an extremely high identity to IS1191 originally identified in S. thermophilus. Furthermore, a region close to the middle of the operon contains DNA sequences with an unusually high identity to genes from the S. thermophilus Sfi6 exopolysaccharide operon. These sequences correspond to the epsA and epsB genes and are probably rearranged/inactive in S. macedonicus as genes corresponding to the homologous function are present and complete at the start of the operon (the usual position).

The bioinformatic analysis identified four of the six sugar-transferase genes, while two identified protein coding regions showed no known sequence similarities. The present invention provides that these additional coding regions encode two additional S. macedonicus exopolysaccharide sugar-transferase genes.

What is claimed is:

1. A biologically pure culture of a lactic acid bacteria strain that comprises a 16S ribosomal RNA comprising a nucleotide sequence that is SEQ ID NO:1 or a homologue thereof having 1-8 nucleotide substitutions, deletions, or additions, and comprising cocci morphology, a growth optimum in the range of about 28° C. to about 45° C., and the ability to ferment D-galactose, D-glucose, D-fructose, D-mannose, and N-acetyl(D)-glucosamine, salicin, cellobiose, maltose, lactose, sucrose and raffinose, and imparts a viscosity of greater than 100 mPa.s at a shear rate of about 293 s⁻¹ when used to ferment semi-skimmed milk at 38° C. at up to a pH 5.2.

2. The strain of claim 1, wherein the 16S ribosomal RNA is SEQ ID NO:1.

3. The strain of claim 1, wherein the strain produces an exopolysaccharide comprising a chain of glucose, galactose and N-acetylglucosamine in a proportion of 3:2:1 respectively.

4. The strain of claim 1, wherein the total protein profile obtained after culture of the bacterium in an MRS medium for 24 h at 28° C., extraction of the total proteins and migration of the proteins on an SDS-PAGE electrophoresis gel, exhibits a degree of Pearson correlation of at least 78 with respect to the profile obtained under identical conditions with the strain of lactic acid bacterium CNCM I-1920 or I-1 926.

5. The strain of claim 1, further comprising a nucleotide sequence that encodes the polypeptides identified by SEQ ID NOS:18, 20, 22-24, 27, 28, 32, and 34 (SM-epsA, C, E-G, J, K, 0, and Q), wherein the strain produces an exopolysaccharide comprising a chain of glucose, galactose and N-acetylglucosamine in a proportion of 3:2:1 respectively.

6. A dietary or pharmaceutical composition comprising a polysaccharide secreted by the strain of claim 1.

7. The composition of claim 6, wherein the polysaccharide secreted has a chain of glucose, galactose and N-acetylglucosamine in a proportion of 3:2:1 respectively.

8. The composition of claim 6, wherein the polysaccharide is hydrolyzed and comprises polysaccharides that have predominantly 3 to 10 sugar units.

9. The composition of claim 7, which is a hypoallergenic infant composition.

10. A dietary or pharmaceutical comprising a strain of lactic acid bacterium according to claim 1.

11. A method of preparing a dietary or pharmaceutical composition comprising: adding a lactic acid bacterium strain according to claim 1 to a dairy product to prepare the composition.

12. The method of claim 11, wherein the dairy product comprises milk.

13. A biologically pure culture of a lactic acid bacteria strain, wherein the bacteria strain comprises nucleotide sequences which encode polypeptides identified by SEQ ID NOS:18, 20, 22-24, 27, 28, 32, and 34 (SM-epsA, C, E-G, J, K, O, and Q), and the strain produces an exopolysaccharide comprising a chain of glucose, galactose and N-acetylglucosamine in a proportion of 3:2:1 respectively.

14. The strain of claim 13, having a total protein profile, wherein the total protein profile obtained after culture of the bacterium in an MRS medium for 24 h at 28° C., extraction of the total proteins and migration of the proteins on an SDS-PAGE electrophoresis gel, exhibits a degree of Pearson correlation of at least 78 with respect to the profile obtained under identical conditions with the strain of lactic acid bacterium CNCM I-1920 or I-1026.

15. The strain of claim 13, wherein the strain further comprises a nucleotide sequence which encodes the polypeptides identified by SEQ ID NOS:21, 25-26, and 33 (SM-epsD, H-I, and P).

16. The strain of claim 15, wherein the strain further comprises a nucleotide sequence which encodes the polypeptides identified by SEQ ID NOS:19 and 29-31 (SM-epsB and L-N).

17. The strain of claim 16, wherein the strain comprises SEQ ID NO:4.

18. A dietary or pharmaceutical composition comprising a polysaccharide secreted by the strain of claim 13.

19. A method of preparing a dietary or pharmaceutical composition comprising: adding a lactic acid bacterium strain according to claim 13 to a dairy product to prepare the composition.

20. The method of claim 19 wherein the dairy product comprises milk.

21. An isolated nucleotide sequence that encodes a peptide identified by SEQ ID NOS:18, 20, 22- 27, 28, 32, 34, or 35 (SM-epsA, C, E-K, O, Q, or R).

22. A transformed microorganism comprising a nucleotide sequence of claim 21, wherein the microorganism produces an exopolysaccharide comprising a chain of glucose, galactose and N-acetylglucosamine in a proportion of 3:2:1 respectively.


SEQUENCE LISTING (37 sequences)

SEQ ID NO: 1 - DNA, 1522 bp, Streptococcus macedonicus
  misc_feature (1460)..(1460): n is a, c, g, or t
gtcgacagagttcgatcctggctcaggacgaacgctggcggcgtgcctaatacatgcaagtagaacgctgaagactttagcttgctagagttggaagagttgcgaacgggtgagtaacgcgtaggtaacctgcctattagtgggggataactattggaaacgatagctaataccgcataatagtgtttaacacatgttagagacttaaaagatgcaattgcatcactagtagatggacctgcgttgtattagctagttggtggggtaacggcctaccaaggcgacgatacatagccgacctgagagggtgatcggccacactgggactgagacacggcccagactcctacgggaggcagcagtagggaatcttcggcaatgggggcaacctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggccgcacaagcggtggagcatgtggtttaattcgaagcaacgcgaagaacttaccaggtcttgacatcccgatgctatttctagagatagaaagtttcttcggaacatcggtgacaggtggtgcatggttgtcgtcagctcgtgtcgtgagatgttgggttaagtcccgcaacgagcgcaacccctattgttagttgccatcattcagttgggcactctagcgagactgccggtgataaaccggaggaaggtggggatgacgtcaaatcatcatgccccttatgacctgggctacacacgtgctacaatggttggtacaacgagtcgcaagccggtgacggcaagcaaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttagganccagccgcctaaggtgggacagatgattggggtgaagtcgtaacaaggtaaccgtaggatcc

SEQ ID NO: 2 - DNA, 34 bp, Streptococcus macedonicus
atatccgttttttcgacagagttygatyctggct

SEQ ID NO: 3 - DNA, 33 bp, Streptococcus macedonicus
atatccggatcctacggytaccttgttacgact

SEQ ID NO: 4 - DNA, 18373 bp, Streptococcus macedonicus
acgccaatttctgaacggaaattcttaacatcatcaataatttcatatgttcgtgtttcgcgaaggaaaagctcataacgtgacatatctgttccttctaaaagcgaaacaaaagcattgacaacgaaagcatagtgctgtgaagatacgctaaacagttctcggtttgtatttttgctcttataacgttcctctaaaagagctgtttgttctaaaatttggcgagcataagaaagaaactcaacgccatccttagtcaaggtaatgccttttgggttacgaataaaaatttcaattcccatttcacgctccaaatctcgtacagcatttgaaaggcttggttgagtgataaaaaagctgcttagcagcctcgttcatgctccctgtttctactattttaacgatatagtgtaattgttgtattctcataggcttagtttagcctaaaaatgaaattcccgcaagtagacaatatcttcttatgacgggagtgctttaaaaacgaatgtttacattacaacaacaaaattacaaaaagataactaaaacgtaacaatttagcgattgatttacttttcttaaaataaaacgcttatttttttaaataatactttaggaagcgcatacagtcgtaaaaattcagaaaattacaaaattgcaaaaaacttacaaaagtgctaaaataggaacgttaatatccttataggaatcggagatttaaaatgtctaaacattcacgtcatagaagacatcataagagttcacgttcatactctcgttttgatacgaagacgatagtgaatagtgttttattagtgttgtttgctttgttagcggggattgcaacttatctcatgtatgccaataatattctagcttttcgtcatctgaatattatctacaccgttttactagttgctgtcttcctcatatctttggttttgataattcggaaaaaagggaaaatcgttgtgacggttctcttggttattttctcgattgttgcagctatttcgctatttgcctttaaatcattggttgatgtggctaatgatatgaataaatcagcctcatattcagaaattgagatgagtgttgtggtgccagcggatagctcaatctcagatgtgacagaattatcaagcgttcaagcaccaacaaatgctgatggtagcaatatcgatactttgctttctcaaattaagtcagataaaggtattgatttagcgacagaaacagtagattcttatcaagccgcttatgaaaatttgattaatgggtcaagtcaagcaatggttttgaacagtgcttattcaagcttgcttgaattatcatataatgattacgaatcaaatttaaagaccatttatacctataaaattaagaagagtgtttcaagcgaagcaaaatcatctgatgctaatgtctttaacatttatattagtggtattgatacctacggatctatttcaaccgtttcacgttcagatgttaatatcatcttgacagttaatatgaatacacataagattttgatgacaacagcaccacgggactcatatgttcaaattccagacggaggtgcagatcaatacgataaactgacacacgctggtatctatggtgttgaaacatctgaaaagacactagaaaatctttatggtattgatattgattattacgctcgtatcaacttcacatcatttatgaatctgattgatgctattggtggtgtgacagtttataatgatcaggcatttacaagtctccatggtaattataattttgaagttggaaatgttaacttaagctcaggtgaagaagcacttgcttttgttcgtgaacgctatagtcttaataatggcgactacgatcgtggtaataatcaaatcaaagttattcaagctattgttaataaattaacatcgttaagttcaatttcaaattactcaacaattatttctaccttgcaggattctattcaaaccgatatgtcattagatacaatgatgagccttgctaatgctcagcttgattcaggtaagaaatttaccattacatcacaagaagtaactggtacaggttcaacaggagaattgacttcttatgccatgccaactgcaagtctttatatgattcagttggatgatgctagtgtagcaagtgcatcacaagccattaaagatgttatggaaggtaagtagatgattgatattcattctcatattgtttttgatgtagatgacggaccaactactattgaagaaagtttagctttggttggggaaagttatcgtcagggcgtgcgtacgattgtctcaacgtcacatcgccgcaaaggaatgtttgaaacaccagaagataagatttttgctaattttagtcaagtcaaagaagctgctgaagccaaatatgaaggcttagaaatcttatatggtggcgaactctactatagtagcgatattctggaaagactggaacaacgccaagttccaagaatgaacgacacacgttttgcattgattgagtttagtatgacaacaccatggaaagagattcatacagcacttagcaatgtgattatgcttggaattacaccagttgttgctcatatcgaacgttataatgcgcttgaatttaatgaagaacgtgttaaagaattgattaacatggggggttacacacaaattaatagctcacatgttctcaaaccaaaattatttggtgataaataccatcaattcaaaaaacgagcacgttatttcttggaaaaaaatcttgttcattgtgtcgcaagcgatatgcataaccttggaccaagaccgccatttatggataaagctagggaaatcgttacaaaagattttggaccaaatagggcatatgctcttttcgaggaaaatcctcaaaccttattagaaaataaagatttataggagttaatatgaattcaaatgataatgcaagtatcgagattgatgtactctacttgctaagaaaactttggagtagaaaatttttcattattttcattgctctagttgttgggacagtagctttgcttggtagtgttttcttcctcaaacctaagtacacatcaacaactcgtatttatgttgtgagccgaagtagtgatggcagcttaactaatcaagatttgcaagcaggttcttatcttgttaatgactataaagaagtcattacgtcaaatgaagttttgtcatctgtcattagtcaagaaaatctctcactttcaacaagtgaattgtcaaatatgatttctgtaaatattccaacagatacacgtgttatttcaatctctgttgaagatacagatgcgaaagaagcttctgatattgctaacactatccgtgaagttgctgcagaaaaaatcaaatctgtaaccaaggtagatgatgtgacaactttggaagctgccgaagtcgctagcaaaccatcatcaccaaatattaaacgcaatgctgctttaggtgtacttgttggtggtttcttggctattgttggtattcttgtgcttgaagtacttgatgaccgtgttcgtcgtccagaagacgtcgaagaagtgcttggtatgacacttttaggagttgtaccagatattgataaattataaggagaaaaattgtaatgccacagttagaattagtgagagctaaagctcaaatggttaaatctatggaggaatattacaattctatccgtaccaatattcaatttagtggacgtgatttaaaagtcattacgttgacttcggctcaatctggcgaaggaaaatcaacaacgtctgttaatcttgcaatttcttttgcgcgtgcaggtttccgtacacttttgattgatgcggatacacgtaactcagtcatgtcaggaacgtttaaatctaaggaacgttatcaggggttgacaagtttcttgtctggaaatgcagagttgtcagatgttatttgtgacacaaatattgataatttgatgattattcctgctgggcaagtcccaccaaaccccacatcattgattcaaaacgataacttcaaagcgatgattgaaattattcgtggactttacgactatgttatcattgatacaccaccgcttggcttggttattgatgcagctatcttagcgcattactcagacgctagcttgcttgtagtaaaagcgggggctgataaacgtcgtacagttacaaaactaaaggaacaattggaacaaagtggttcagctttccttggcgttattctgaataaatatgatattcaggtagtgtaaaataagttgtgtaaacacaaaaaggaataaatccgttatagtagagttgcaaaacattactagaaagagatttattcctatgactcagtttaccacagaactacttaacttcctagcccaaaagcaagatattgatgaatttttccgtacttctcttgaaacagctatgaatgatctgcttcaagcagagttatcagcctttttagggtatgaaccttacgataaattaggctataattctgggaatagtcgtaacggaagctatgcacggaaattcgaaaccaaatatgggactgttcagttgagtattcctagagatcgtaatgggaactttagtccagctttgcttcccgcttatggacgtcgagatgaccacttggaagagatggttatcaaactctatcaaaccggtgtaacgactcgagaaattagtgatatcatcgagcgaatgtatggtcatcactatagtcctgccacaatttctaatatctcaaaagcaactcaggagaatgtcgctacttttcatgagcgaagcttagaagccaattactctgttttatttcttgacggaacctatcttccattaagacgtggaaccgttagtaaagaatgtattcatatcgcacttggcattacaccagaaggacagaaggctgttcttggatatgaaatcgccccaaatgaaaacaatgcttcttggtccaccctgttagacaagcttcaaaaccaaggaatccaacaggtttctcttgtagtgaccgatggcttcaaggggcttgaagagattatcaatcaggcttacccattagctaaacaacaacgttgcttaattcatattagtcgaaatctagctagtaaagtgaaacgagcagatagagcggttattctggagcaatttaaaacgatttatcgtgctgaaaatttagaaatggcagtgcaagctttagagaactttatctccgaatggaaaccaaagtataggaaagtcatggaaagtctggagaatacggataatcttttaactttttatcagtttccctaccagatttggcatagtatttattcgacaaacctcattgagtctcttaacaaagagatcaaacgtcaaacgaaaaagaagattctttttcctaacgaggaggctctgggacgttatttagttaccctgtttgaagattataatttcaagcaaagtcaacgcacccataaagggtttggccaatgtgctgacacacttgaaagcttatttgattaacattcttcaactctacttgagtgtttacacataattattgacagtatcgatattcacttagataagtatggttcatatggtagttacggtgggtatggtagttatggcaattacggaaaaagtgaagaaaaaacaaaaattggtagaggtaacgaaaaaaatagctgatacttttaccttagaatagggaacagggagttacatgtatagcgaagattcgaaaaagaaagtttattaccttttgtcggatattatagccttagtgataagttacctcatcttagcacaattttatccttatcatttttttgatagtaaattctttgcagttgtttttgggattctgattgtgattgttagtgttttgagtgatgaatactcttcaattaaaaatcgtggttatttaaaagaattaaaagcatctgtgatttatggtatgaaagttttagttttatttacttttgtactgatacttggaaaaattcgttttatccatgacatttcacagatgtcttatttcttattggggcaaatttttattttagtaagcctttttgtcttcattggacgtattttagttaagaatcttttcagaagtcatgcaacggatattaaacaggtagtgtttgtcacggattttacgaatggtaaggaagtcattaaagagcttagcaattccaattaccatatcgctgcttatatcagtcgtcgtgataatcctgatatttcacagcctatcttaaaaagtactaaagaaattagggattttgtggcaaatcaccaagttgacgagatatttgttgccaaaaatcaccaagatgattttattgaatttgctcattgcttaaaattgttaggaattccaacgacagtagctgttgggaattattcggacttctatgttggaaatagtgttctaaaaaaagtaggtgatacgaccttcataacgacagcattcaatattgtaaaattccgtcagattgctttaaaacgtcttatggatattgcaatagctttagttggcttagtgattactggtattgtagccattattatcacaccgataatcaagaaacaatcaccaggacctctaatcttcaaacaaaaacgtgttggtaaaaacggtaaagtttttgaaatttacaaatttagaagcatgtacaccgatgccgaagaacgcaaaaaagaattactaacacaaaatgatttggatactgacttaatgtttaagatggatgatgaccctcgtatcttcccatttggacataagttacgtgattggtcacttgatgaattaccacaatttattaatgtcctaaaaggtgaaatgtctgttgtgggcacacgtccaccaacgcttgacgaatatcatcactatgagttacatcatttcaaacgattgacaaccaaaccaggaattactggtttatggcaagttagcggtcgtagtgacattaccgactttgaagaagtcgtagcacttgatatgaagtatatccaaaactggagcatcagtgaagatattaaaattattgccaaaacatttggagtcgtactaaaaagagagggaagtaagtagagtatattatgaaagtttgtttagtaggttcttctggtggacatttggcacatttgaatatgctaaaacccttttggagtgaacatagccgtttccgggttacatttgataaagaagacgcaagaagtgtgttaagtgatgaaaaattttatccgtgttattttccgactaacagaaattttaagaatttggtaaagaacactttcttagcacttgaaattttaagaaaagaaaaacctgacgttattatttcatcaggagcagcggtagcagttccatttttttatctgggtaaactgtttggagcgaaaacggtttatatcgaagtatttgatagaatagataaaccgactgtgactggaaagttggtttatccagtgacagataaatttattgttcagtgggaggagatgaaaactgtctatcccaaagctattaatctggggagtattttttaatgatttttgttacagttggaactcatgaacagccctttaataggcttattaaggaagttgatcgtttaaaaaaagaaggtattattacagatgaggtttttattcagacaggtttttcaacttatgagcctcaatactgtgactggaaaaatattatttcttattctgaaatggaagattacatgaatcgtgcagatattattatcacgcatggtggtccagcgacattcatgggagcaattgctaaaggaaaaaaaccgattgttgttccaagacaggaaaagtttggagagcatgtaaatgatcatcagcttgagtttgctgaacaggtttctgaacgatttggaagtatcgttgtcgtagaagaaattaatgaattgcaaaattattttaatttagatttaattgtagatgaaagttccaattcgaacaacctaagatttaatagtcaattaaaacaagaaatagaaagtttggttagatgaatgattcctaaaaagattcattattgttggtttggaggaaatcctcttcctgacagtgtaaaaaattgtataaattcgtggaaaaaattctgtccaaattatgaaataatcgaatggaatgaatcaaattatgatgtacataaaattccatatatttctgaagcttataaaaataagaaatatgcttttgtatctgactatgctaggctagatatcatatataatgagggcgggttttatttagatactgatgttgaattgttaaaagcattggacgatttaacttctgaacactgttatatgggaatggaacaagtgggtcgtgttaatactggattaggttttggtgcagaaaaaggacatctttttataaaagaaaatatgcagcaatatgaagaagtttcttttaatcttaagctactagaaacatgtgtggatatcacgacaaatttattattatcaaaggggttattagtagaaaattcatatcaaaaaattagtgatgtgtcaatttatccaacagattttttttgtccgtttaatatgcaaacacaagaaatgggaataactaaaaatacttattcaattcatcattatgattcaacttggtatggtaatggtgttagtgcaataattaaaaagaaattattaccattaagagttaaatctcgtatccttattgataaatatttaggtgaaggctcttatgctaaaatcaaagctattattaagaaatgatatttttcaaaggaggatattttgttaactaatattgaattttttgatatatatatatttcttgttactctatttaaaggattgggagctgaagcaggtaataaattatatgttgtagcattttttataggatctattgcgatttgtttaaaaatttcaaaggaaaaattttcatttaatgaacttaaaaaagttacttttattttgataatagggctattagattttattgttggcaaaagtacaacgtttttgtttactgcaattgcattaagtggacttaaaaatgttaatgaaaatcgagttatcaaaattgctttttggactagattattctcttttctattaatggtgagtctaagtaaattgaatattattaaagataacttgttccttttttatagggatggccagtttgtaggaaggcatacatttggttatggacatccgaatcaagcgcagagtgctttaacaattttgataatacttgctatttatctttataatgagaagtttaatattttccattatatcattatgattattatgaacttctatttatatagcttaacatattcgcgtacaggtttcttgatcggagtattatgtattgttctgggagtggttcaaaaaagtaaaaatgtagaaaaaatttttgctagagtatttaaaaactcatatttttgggctgttttagtgacgctatttatagggtatttttacactaagattccacaattaaaaaacttagatgaattattcacaggtaggttggcttataacaacactttattaaataattatattccgccacttattgggagttcaaaatacaatgagtatgttaatatcgataacggttttatttctttgatatatcaaggaggtattttagcatttttgtggatttcggcttgtatcataaaattaatgaatgatttttatatccaaaaaaaatttagggagttgttttttatgagcagctttatagtttatggaatgacagaaagtttttttccaaatattgctgttaatatctctcttattttcattggtaaactgatatttaaaactcgcgaggaagttatgaatgcataaagtttttatttttacaccgacatacaatagagtggaaaatctaaagaaattgtatgagtcactaaggaagcagacttgtaaagagtttatttggctaattgtcgatgatggttcaaatgatggtactgaattttatattagacagttacgatctgaatatatttttgatattgtatacctaaaaaaagaaaatggaggcaaacatactgcgtataatttagctttagattatatgggaggagagggatggcatatggttgtagatagcgatgattggttagctagcacagctgttgaatgtattattaaagatatctcctcacttcaagttggtaagcttggagttgtatatccaaaatatagtttaactgaagaattacgatggttacctgagaaagtaactgaagttaatattccagacataaaattgaaatacgggctttcaatcgagactgcaattgttattaaaaatttattcattggtcaattgagacttccttcatttgagggggagaagtttttgtctgaagaaattttttatattatgctatcggagtttggaaaatttcttcctcttaatagaagaatatatttttttgaatatctagaacatggtctaactaataatctttttcatctgtggaagaagaaccccaagagcacttatttattgtttaaagagagaaaaaaatatatcctgcaaaatttatcaggttttaaccgaattgttgaattgtttaaagtgtccttgaatgaacaagcattatcgctagcaacatcaaagaatgaaaatattccccaagagctatctgttggggaacgtatgctaaaaccattggcatatttattttatttaaaaaggtataaataggaataagtatcatagtgaggagatattgtggataatgagttaatcagtattattgttcctgtatacaatgttgaaaaatacattgctaagtgtttggactctttagttaaccaaacatatttaaatatagaaatacttctaattgatgatggatctacagacaaatcattatcgatatgtaagaagtatgctgcagttgattctcgaattaagcttttttctaaagagaatggcggcgtttctagcgctcgaaatctaggtcttctacatgttcaaggagagtacgttgtgtttgtagattcagatgactttgtatcaccaaaatattgtgaacatttatatcaacttactataagtactaagtcagagttagcttctgtaagtcgttataacattttgaataaagaggtggtaaagatatcggatttatcttttaatcaaataacatcagatgaagccttaagaaaattctttttaggtgaggggataaattgttatcttttttcaaaaatatttaaatatgaaactataaaaggactccgatttgatgaaagtttagaatcagcagaggacgttttgtttatttatcaaactcttaagaacataaattttgcatctatggatggcactgttgcagattatttttatattcttagagaaggatctttaacaaataaaagactgacttcatcaagaattgatagttccattagagttgcggaatttattactagagattgcaacagcaacaaaaaattgaaaatgttaagtgaaattaatgaaatatcattaaagggtgaggttcttgagtggatttcattaaatagtgaacttagaattgagtttgaagaatattataatatcatactgagagaagttagaaagtttaaattgttacataaagttcaatatctaactttaaaaaaatttattaggattatattattaaaagttagtcctagattagttacaatcttaaaaaataaataggtatcctggaaggagtattcatggattttaatagtaaccctcttgtttcaattattattccaatttataatgtagaaaattatttagaacagtgctctacttgagtgtttacacataattattgacagtatctcacaatataatggaaaatgatataaattaaatgattgatatcataataaaaactttttcttatgttttgaaaaaagaatgacaattgaaatgaagttgtattaatgttatattaataataatgggggatatctaattttaatttttaggagcaatttatatgagttcgcgtacgaatcgtaagcaaaagcatacgagtaatggatcgtggggggatggtcaacgttgggttgaccattctgtatgctattttagcattggtcttattattcaccatgttcaattataatttcctatcctttaggtttttgaacatcattatcaccattggtttgttggtagttcttgctattagcatcttccttcagaagactaagaaatcaccactagtgacaacggttgtactggttatcttctcgctagtttctctggttggtatttttggttttaaacaaatgattgatatcactaaccgtataaatcagactgcagccttttcagaagtagaaatgagcattgtggttccgaaggatagtgacatcagagatgtgagtcagattactagcgttcaggcaccaactaaggttgataagaataatatcgatagtttgatgtcagctctaaaggaagacaaaaaagttgatgacaaagttgatgatgtcgcttcctatcaagaagcctatgacaatcttaagtctggcaaatctaaagctatggtcttgagtggctcttatgctaccctattagagtctgtcgatagtaattatgcttcaaatctaaaaacaatttatacttataaaattaaaaagaaaaatagcaactctgcaaaccaagtagattcaaaagtcttcaatatttatattagtggtattgatacctacggtccgatttcaacagtatcacgttcagatgtcaatatcattatgacagtaaacatgaatacacataagattctcttgacgactactccacgtgatgcatacgttaagattgggcagaccagtatgataaattaacccacgcaggtatttatggcgttgaaacatctgaacaaactctggaagatctttatggtattaagattgattactatgcacgaattaacttcacatctttccttaagttgattgaccaacttggtggtgtgacagtccataatgatcaagctttcacacaagggaagtttgatttcccggttggagatatccaaatgaattcagagcaagcacttggatttgttcgtgaacgctataatttagatggcggagataatgaccgtggtaaaaaccaggagaaagttatttctgcgattttaaacaagttggcttctctaaaatctgtatcaaactttacttcaatcgttaataatctccaagactctgttcaaacgaatatgtctttgaatcccattaacgctttggctaatacacaacttgaatcaggttctaaatttacggtgacttctcaagcagtaacaggtacaggttcaaccggacaattgacctcttatgcgatgccaaattctagtctttacatgatgaaactagataattcgagtgtggaaagtgcctctcaagctatcaaaaaattaatggaggaaaaataagtgattgacgttcactcacatatcgtttttgatgttgatgatggtcctaaaactttagaagaaagtttagacctcattggtgaaagttacgcccagggggtacgtaagattgtttcaacatcccatcgtcgtaagggaatgtttgagactccagagaataaaatttttgccaacttttctaaggtaaaagcagaagcagaagcactttatccagacttaactatttattatggaggtgaacttgattataccttggacattgtggagaaacttgaaaagaatctcattccgcgcatgcacaacactcaatttgctttgattgagtttagtgctcgcacatcttggaaagaaattcatagtgggcttagtaatgttttgagagcgggggtaacacctattgttgctcatattgagcgctatgatgccctcgaagaaaatgctgaccgtgttcgagaaatcatcaattacgacactaggaattgcaagtaaaaatgggagagtagaatgaaagttttaaaaaattacgcctacaatctttcctatcaattactggtcattgttttaccaatcattacgacaccttatgttactaggatttttagttcaaaggatttaggtacttatggttactttaattcgattgtggcctactttattcttttggcaactttaggtgttgctaactatggtactaaagagatttcaggacatcgaaaggatattcgtaaaaatttctggggtatttataccctccaattgattgcgactattttgtctcttgtcttgtatacatcattatgtttattctttcctggtatgcaaaatatggtggcttatatcttaggattaagcttgatatcgaaaggaatggatatttcttggttattccaaggtttggaggattttcgtcgtattaccgcaaggaatacaacggtaaaggttttaggagttatttctatcttcctatttgtgaaaacacctggtgatttgtatctctatgttttcctattgaccttctttgaattgcttgggcaattaagtatgtggttaccagcgagaccttacattggaaaaccacaatttgatttatcctatgctaagaaacgtcttaaacctgttattttgctgtttctccctcaggttgccatttcactatacgtgactttggatcgtacaatgttgggtgccttgtcatcgacaaatgatgtagggatttatgatcaggctttgaaaataattaatattttgttgacgttggtgacttcattgggaagtgtaatgcttccaagggtatctggtcttttatctaacggagatcataaggccgttaacaagatgcatgagttgtctttcttgatttataatcttgtgatcttcccgataatagcaggtctcttgattgttaataaggattttgtgagtttcttcctagggaaagatttccaagaggcttatcttgccattgctattatggtctttaggatgttctttatcggttggacaaatattatgggaatccagattttgattccacacaataaacatcgtgagtttatgctctctacgactattccggctgttgtcagtgttggacttaatctcttgttaattcctccatttggcttcgttggtgcctcaattgtatcagttttaacagaggctttggtatggttcattcaattgtacttctgccttccttacctcaaggaagtaccgattcttgagtctttggccaaaattgtatgcgcatctactatgatgtatggcttgttgctaagtgcaaaaccattcttgcattttccacctactttaaatgttcttgtgtatgcagtgattggtggcctcatttaccttcttgctattctagttttgaaagtggtagatgttaaagaattaaaacaaataataggagaaaattaggaatgaagaaagcacggaatataaacttagacttgataaaaataattgcttgtataggagttgttttgcttcatactacgatgccagggtttaaggaaacagggcgatggaattactcatcttatttatattatctaggtacttattaaattaccttgttttttatggtaaatggttatttattattgggtaagagcaagataacatatccctatatactacataaaataaaatggtttctaataacagtgtcttcatggaccgttatcatttggtttcttaaaagagacttcacaattaatccaattaaaaaaattttggcttccttgatacaaaagggttatttcttccaattttggtttttcggatcactaatacttatttatttatgcttgccgatattgaagaagtatttacattcaaaaagaagttatttatactttctatatgtattaacaattattggtttgatttttgaattgataaattttttgcttcaaatgccagtacaaatttatgttatacagacgtttagattatggacttagttcttttactacattttaggtggttttgtagcacaattcaatatagagaatttaaaatcaatctttaagggatggatgaaaatagttagcatacttttgttattgatttcaccgataatattatttttcatagcaaaaactacttatcataatctttttgctgaatatttttatgacaatcttttggtaaaagtaattagtttaggactatttcttaccttattgacgctaaccattgatgcttctaaacatagaatgatctacttgttatcagtccaaacgatgggggtatttatcatacatacctatgttatgcaaatatggcaaaagttgatagggtttaacatagtaggtgcacacttatttttccctgttttcacattagtgattagttttctaataagtatgatattaatgaaaatcccttatatcaatcgaatagttaaattataaaaaggagtttataatgtacgattatcttattgttggtgctggtttgtccggagcaatcttcgcacaggaagctacaaaacgtggcaaaaaagtaaaagtgattgacaagcgtgatcacattggtggcaatatctactgtgaagatgttgaaggtattaacgttcacaagtatggtgctcacattttccatacctcaaataaaaaagtttgggattatgtcaaccaatttgctgaatttaataactatatcaactcaccaattgctaactacaagggcagtctttataaccttccatttaacatgaatacattttatgctatgtggggcactaagactcctcaagaagttaaggacaagattgctgagcaaacggctgatatgaaagatgttgagcctaaaaacttggaagaacaagctatcaagttgattggaccagatatctacgaaaagttgatcaagggatacactgaaaaacaatggggacgttctgcgacagacctgcctcctttcatcatcaagcgtcttccggttcgtctgacttttgataacaactactttaatgaccgttaccaaggaattccgatcggtggttacaatgtcatcattgaaaatatgcttggagatgttgaagtagaacttggagttgacttctttgccaatcgtgaagagcttgaagcttcagctgaaaaagttgtctttacaggaatgattgaccagtactttgattataaacatggtgagttggagtatcgcagtcttcgttttgaacacgaagtcttggatgaagaaaatcatcaaggaaatgccgtggtcaactacacagagcgtgagattccttatactcgtatcattgagcacaagcacttcgagtatggtacacaacctaagacagttatcacacgtgaatacccagctgattggaaacgaggagatgaaccatactacccaatcaatgatgaaaagaacaatgccatgtttgctaagtaccaagaagaagctgagaaaaatgacaaggttatcttctgtggacgtcttgcagattataaatactacgacatgcacgtggtcattgagcgtgctctagaagtcgttgagaaagaatttactatatgacacaagaaaaaaattgatatcgttgttctttgggtagatggaagtgccccagagtttatccgtgagaaacaagcagttactgagaatgtttctgatttgaaccaagaaattgatggtgagcaacgttatcgtgattatgatgtttttaattactggttccgaatgattgaaaagaatgctccttgggtaaataatgtctatttgattaccaatgggcaaaagccagactggttgaatttggaacatccaaaactcaaattggtaactcatagggaatttatgcccaaagaatacctaccgacctataattcagcagctattgagcttaatcttcatcatattgaagggttgtcggagaactacttgtatttcaatgatgatacgtacttgattagagacagtcaaccttcagatttttataaaaatggtcagcctaagcttttagctgtttatgatgccttagttccttggccaccatttacgaatacttatcacaataatgttgaattaatttatcgccattttcctaataagaaggctttgaagtcttcgccatggaaattctttaatttccgttatggttccttggttttgaaaaacttgttactcttgccttggggtcctacgagatacgtgaaccagcatttacctgttccgatgaagaagagtaccttggcacatttatgggaaattgaaggtgaaactttagataaaacatcgcgaaatccaattagagactatggagtagatgttaatcaatacatctgtcagcattggcaaattgaaagtaaccagttttaccctatgtctaaaagtttcggagagacaatcggttt

SEQ ID NO: 5 - protein, 100 aa, Streptococcus macedonicus
MGIEIFIRNPKGITLTKDGVEFLSYARQILEQTALLEERYKSKNTNRELFSVSSQHYAFVVNAFVSLLEGTDMSRYELFLRETRTYEIIDDVKNFRSEIG

SEQ ID NO: 6 - DNA, 480 bp, Streptococcus macedonicus
tgcggttaaagacttgcctttaagaattgtagtagttattaaagtatacaagcacaaagcgcttccttttcgagtattgcactgtatagacaaggaagattttcgctttgttttcgtaactgttgctttcgtatcacgacacttctatgcgatttgtcaagagccaaacataaaaacgagaatattgcaaggagattttctcgacaaacaagattttaaaccgctcgtattctttctttgagttgcggtaggaatcagttccattacggaaaacccaatgcttatttttaaagttaagggtaaagtgcgaggtttagagcatgtcgtaaactttccgaaccaactcactattttttcgacgaatcgtcggagcaagtacgagggacaaagatgataaaattgctatatcacattaacaacataagagtatccgaatcaaatcggatttttactttaagggcgttcatctgttatagaaga

SEQ ID NO: 7 - DNA, 20 bp, Streptococcus thermophilus
atgagttcgcgtacgaatcg

SEQ ID NO: 8 - DNA, 20 bp, Streptococcus thermophilus
atacagattttagagaagcc

SEQ ID NO: 9 - DNA, 21 bp, Streptococcus thermophilus
ctgcaaggcgattaagttggg

SEQ ID NO: 10 - DNA, 21 bp, Streptococcus thermophilus
gttgtgtggaattgtgagcgg

SEQ ID NO: 11 - DNA, 27 bp, Streptococcus macedonicus
acaggtaccttgtctggaaatgcagag

SEQ ID NO: 12 - DNA, 27 bp, Streptococcus macedonicus
ctcggatccaaccgctctatctgctgc

SEQ ID NO: 13 - DNA, 27 bp, Streptococcus macedonicus
tccggtacctttctcttgtagtgaccg

SEQ ID NO: 14 - DNA, 27 bp, Streptococcus macedonicus
cgtggatcccgtgacaaacactacctg

SEQ ID NO: 15 - DNA, 1187 bp, Streptococcus macedonicus
tatgagttcgcgtacgaatcgtaagcaaaagcatacgagtaatggatcgtggggggatggtcaacgttgggttgaccattctgtatgctattttagcattggtcttattattcaccatgttcaattataatttcctatcctttaggtttttgaacatcattatcaccattggtttgttggtagttcttgctattagcatcttccttcagaagactaagaaatcaccactagtgacaacggttgtactggttatcttctcgctagtttctctggttggtatttttggttttaaacaaatgattgatatcactaaccgtataaatcagactgcagccttttcagaagtagaaatgagcattgtggttccgaaggatagtgacatcagagatgtgagtcagattactagcgttcaggcaccaactaaggttgataagaataatatcgatagtttgatgtcagctctaaaggaagacaaaaaagttgatgacaaagttgatgatgtcgcttcctatcaagaagcctatgacaatcttaagtctggcaaatctaaagctatggtcttgagtggctcttatgctaccctattagagtctgtcgatagtaattatgcttcaaatctaaaaacaatttatacttataaaattaaaaagaaaaatagcaactctgcaaaccaagtagattcaaaagtcttcaatatttatattagtggtattgatacctacggtccgatttcaacagtatcacgttcagatgtcaatatcattatgacagtaaacatgaatacacataagattctcttgacgactactccacgtgatgcatacgttaagattgggcagaccagtatgataaattaacccacgcaggtatttatggcgttgaaacatctgaacaaactctggaagatctttatggtattaagattgattactatgcacgaattaacttcacatctttccttaagttgattgaccaacttggtggtgtgacagtccataatgatcaagctttcacacaagggaagtttgatttcccggttggagatatccaaatgaattcagagcaagcacttggatttgttcgtgaacgctataatttagatggcggagataatgaccgtggtaaaaaccaggagaaagttatttctgcgattttaaacaagttggcttctctaaaatctgtatcaa

SEQ ID NO: 16 - DNA, 1196 bp, Streptococcus thermophilus
tatgagttcgcgtacgaatcgtaagcaaaagcatacgagtaatggatcgtgggggatggtcaacgttgggttgaccatcctgtatgctattttagcattggtcttattattcaccatgttcaattataatttcctatcctttaggtttttgaacatcattatcaccattggtttgttggtagttcttgctattagcatcttccttcagaagactaagaaattaccactagtgacaacggttgtactggttatcttctcgctagtttctctggttggtatttttggttttaaacaaatgattgacatcactaaccgtatgaatcagacagcagcattttctgaagtagaaatgagcatcgtggttcctaaggaaagtgacatcaaagatgtgagccagcttactagcgtacaggcacctactaaggttgataagaacaatatcgagatcttgatgtcagctctcaaaaaagataaaaaagttgatgttaaagttgatgatgttgcctcatatcaagaagcttatgataatctcaagtctggcaaatctaaagctatggtcttgagtggctcttatgctagcctattagagtctgtcgatagtaattatgcttcaaatctaaaaacaatttatacttataaaattaaaaagaagaatagcaactctgcaaaccaagtagattcaagagtcttcaatatttatattagtggtattgatacctacggtccgatttcaacagtgtcacgttcagatgtcaatatcattatgacagtaaacatgaatacacataagattctcttgacgactactccacgtgatgcatacgttaagattcctggtggtggggcagaccagtatgataaattaacccacgcaggtatttatggcgttgaaacatctgaacaaactctagaagatctttatggtattaagcttgattactatgcacgaattaacttcacatctttccttaagttgattgaccaacttggtggtgtgacagtccataatgatcaagctttcacacaagagaagtttgatttcccggttggagatatccaaatgaattcagagcaagcacttggatttgttcgtgaacgctataatttagatggcggagataatgaccgtggtaaaaaccaggagaaagttatttctgcgattttaaacaagttggcttctctaaaatctgtatcaa

SEQ ID NO: 17 - protein, 332 aa, Streptococcus pneumoniae
MSKFRNINLDLLKVLACVGVVLLHTTMGGFKETGAWNFLTYLYYLGTYSIPLFFMVNGYLLLGKREITYSYILQKIKWLLITVSSWTFIVWLFKRDFTENLIKKIIGSLIQKGYFFQFWFFGALILIYLCLPILRQFLNSKRSYLYSLSLLMTIGLIFELSNILLQMPIQTYVIQTFRLWTWFFYYLLGGYIAQFTIEEIESRFKNWMKIVSILLLLISPIILFFIAKTIYHNLFAEYFYDTLFVKVSTLGIFLTILMLTLNENRRESIVSLSNQTMGVFIIHTYIMKVWEKVLGFNFVGAYLLFALFTLSVSFIIVGMLMKIPYFNRIVKL

SEQ ID NO: 18 - protein, 493 aa, Streptococcus macedonicus
MSKHSRHRRHHKSSRSYSRFDTKTIVNSVLLVLFALLAGIATYLMYANNILAFRHLNIIYTVLLVAVFLISLVLIIRKKGKIVVTVLLVIFSIVAAISLFAFKSLVDVANDMNKSASYSEIEMSVVVPADSSISDVTELSSVQAPTNADGSNIDTLLSQIKSDKGIDLATETVDSYQAAYENLINGSSQAMVLNSAYSSLLELSYNDYESNLKTIYTYKIKKSVSSEAKSSDANVFNIYISGIDTYGSISTVSRSDVNIILTVNMNTHKILMTTAPRDSYVQIPDGGADQYDKLTHAGIYGVETSEKTLENLYGIDIDYYARINFTSFMNLIDAIGGVTVYNDQAFTSLHGNYNFEVGNVNLSSGEEALAFVRERYSLNNGDYDRGNNQIKVIQAIVNKLTSLSSISNYSTIISTLQDSIQTDMSLDTMMSLANAQLDSGKKFTITSQEVTGTGSTGELTSYAMPTASLYMIQLDDASVASASQAIKDVMEGK

SEQ ID NO: 19 - protein, 243 aa, Streptococcus macedonicus
MIDIHSHIVFDVDDGPTTIEESLALVGESYRQGVRTIVSTSHRRKGMFETPEDKIFANFSQVKEAAEAKYEGLEILYGGELYYSSDILERLEQRQVPRMNDTRFALIEFSMTTPWKEIHTALSNVIMLGITPVVAHIERYNALEFNEERVKELINMGGYTQINSSHVLKPKLFGDKYHQFKKRARYFLEKNLVHCVASDMHNLGPRPPFMDKAREIVTKDFGPNRAYALFEENPQTLLENKDL

SEQ ID NO: 20 - protein, 229 aa, Streptococcus macedonicus
MNSNDNASIEIDVLYLLRKLWSRKFFIIFIALVVGTVALLGSVFFLKPKYTSTTRIYVVSRSSDGSLTNQDLQAGSYLVNDYKEVITSNEVLSSVISQENLSLSTSELSNMISVNIPTDTRVISISVEDTDAKEASDIANTIREVAAEKIKSVTKVDDVTTLEAAEVASKPSSPNIKRNAALGVLVGGFLAIVGILVLEVLDDRVRRPEDVEEVLGMTLLGVVPDIDKL

SEQ ID NO: 21 - protein, 213 aa, Streptococcus macedonicus
MPQLELVRAKAQMVKSMEEYYNSIRTNIQFSGRDLKVITLTSAQSGEGKSTTSVNLAISFARAGFRTLLIDADTRNSVMSGTFKSKERYQGLTSFLSGNAELSDVICDTNIDNLMIIPAGQVPPNPTSLIQNDNFKAMIEIIRGLYDYVIIDTPPLGLVIDAAILAHYSDASLLVVKAGADKRRTVTKLKEQLEQSGSAFLGVILNKYDIQVV

SEQ ID NO: 22 - protein, 458 aa, Streptococcus macedonicus
MYSEDSKKKVYYLLSDIIALVISYLILAQFYPYHFFDSKFFAVVFGILIVIVSVLSDEYSSIKNRGYLKELKASVIYGMKVLVLFTFVLILGKIRFIHDISQMSYFLLGQIFILVSLFVFIGRILVKNLFRSHATDIKQVVFVTDFTNGKEVIKELSNSNYHIAAYISRRDNPDISQPILKSTKEIRDFVANHQVDEIFVAKNHQDDFIEFAHCLKLLGIPTTVAVGNYSDFYVGNSVLKKVGDTTFITTAFNIVKFRQIALKRLMDIAIALVGLVITGIVAIIITPIIKKQSPGPLIFKQKRVGKNGKVFEIYKFRSMYTDAEERKKELLTQNDLDTDLMFKMDDDPRIFPFGHKLRDWSLDELPQFINVLKGEMSVVGTRPPTLDEYHHYELHHFKRLTTKPGITGLWQVSGRSDITDFEEVVALDMKYIQNWSISEDIKIIAKTFGVVLKREGSK

SEQ ID NO: 23 - protein, 149 aa, Streptococcus macedonicus
MKVCLVGSSGGHLAHLNMLKPFWSEHSRFRVTFDKEDARSVLSDEKFYPCYFPTNRNFKNLVKNTFLALEILRKEKPDVIISSGAAVAVPFFYLGKLFGAKTVYIEVFDRIDKPTVTGKLVYPVTDKFIVQWEEMKTVYPKAINLGSIF

SEQ ID NO: 24 - protein, 161 aa, Streptococcus macedonicus
MIFVTVGTHEQPFNRLIKEVDRLKKEGIITDEVFIQTGFSTYEPQYCDWKNIISYSEMEDYMNRADIIITHGGPATFMGAIAKGKKPIVVPRQEKFGEHVNDHQLEFAEQVSERFGSIVVVEEINELQNYFNLDLIVDESSNSNNLRFNSQLKQEIESLVR

SEQ ID NO: 25 - protein, 245 aa, Streptococcus macedonicus
MIPKKIHYCWFGGNPLPDSVKNCINSWKKFCPNYEIIEWNESNYDVHKIPYISEAYKNKKYAFVSDYARLDIIYNEGGFYLDTDVELLKALDDLTSEHCYMGMEQVGRVNTGLGFGAEKGHLFIKENMQQYEEVSFNLKLLETCVDITTNLLLSKGLLVENSYQKISDVSIYPTDFFCPFNMQTQEMGITKNTYSIHHYDSTWYGNGVSAIIKKKLLPLRVKSRILIDKYLGEGSYAKIKAIIKK

SEQ ID NO: 26 - protein, 249 aa, Streptococcus macedonicus
MVSLSKLNIIKDNLFLFYRDGQFVGRHTFGYGHPNQAQSALTILIILAIYLYNEKFNIFHYIIMIIMNFYLYSLTYSRTGFLIGVLCIVLGVVQKSKNVEKIFARVFKNSYFWAVLVTLFIGYFYTKIPQLKNLDELFTGRLAYNNTLLNNYIPPLIGSSKYNEYVNIDNGFISLIYQGGILAFLWISACIIKLMNDFYIQKKFRELFFMSSFIVYGMTESFFPNIAVNISLIFIGKLIFKTREEVMNA

SEQ ID NO: 27 - protein, 292 aa, Streptococcus macedonicus
MHKVFIFTPTYNRVENLKKLYESLRKQTCKEFIWLIVDDGSNDGTEFYIRQLRSEYIFDIVYLKKENGGKHTAYNLALDYMGGEGWHMVVDSDDWLASTAVECIIKDISSLQVGKLGVVYPKYSLTEELRWLPEKVTEVNIPDIKLKYGLSIETAIVIKNLFIGQLRLPSFEGEKFLSEEIFYIMLSEFGKFLPLNRRIYFFEYLEHGLTNNLFHLWKKNPKSTYLLFKERKKYILQNLSGFNRIVELFKVSLNEQALSLATSKNENIPQELSVGERMLKPLAYLFYLKRYK

SEQ ID NO: 28 - protein, 320 aa, Streptococcus macedonicus
VDNELISIIVPVYNVEKYIAKCLDSLVNQTYLNIEILLIDDGSTDKSLSICKKYAAVDSRIKLFSKENGGVSSARNLGLLHVQGEYVVFVDSDDFVSPKYCEHLYQLTISTKSELASVSRYNILNKEVVKISDLSFNQITSDEALRKFFLGEGINCYLFSKIFKYETIKGLRFDESLESAEDVLFIYQTLKNINFASMDGTVADYFYILREGSLTNKRLTSSRIDSSIRVAEFITRDCNSNKKLKMLSEINEISLKGEVLEWISLNSELRIEFEEYYNIILREVRKFKLLHKVQYLTLKKFIRIILLKVSPRLVTILKNK

SEQ ID NO: 29 - protein, 271 aa, Streptococcus macedonicus
MDRGGMVNVGLTILYAILALVLLFTMFNYNFLSFRFLNIIITIGLLVVLAISIFLQKTKKSPLVTTVVLVIFSLVSLVGIFGFKQMIDITNRINQTAAFSEVEMSIVVPKDSDIRDVSQITSVQAPTKVDKNNIDSLMSALKEDKKVDDKVDDVASYQEAYDNLKSGKSKAMVLSGSYATLLESVDSNYASNLKTIYTYKIKKKNSNSANQVDSKVFNIYISGIDTYGPISTVSRSDVNIIMTVNMNTHKILLTTTPRDAYVKIGQTSMIN

SEQ ID NO: 30 - protein, 131 aa, Streptococcus macedonicus
MNSEQALGFVRERYNLDGGDNDRGKNQEKVISAILNKLASLKSVSNFTSIVNNLQDSVQTNMSLNPINALANTQLESGSKFTVTSQAVTGTGSTGQLTSYAMPNSSLYMMKLDNSSVESASQAIKKLMEEK

SEQ ID NO: 31 - protein, 162 aa, Streptococcus macedonicus
VIDVHSHIVFDVDDGPKTLEESLDLIGESYAQGVRKIVSTSHRRKGMFETPENKIFANFSKVKAEAEALYPDLTIYYGGELDYTLDIVEKLEKNLIPRMHNTQFALIEFSARTSWKEIHSGLSNVLRAGVTPIVAHIERYDALEENADRVREIINYDTRNCK

SEQ ID NO: 32 - protein, 471 aa, Streptococcus macedonicus
MKVLKNYAYNLSYQLLVIVLPIITTPYVTRIFSSKDLGTYGYFNSIVAYFILLATLGVANYGTKEISGHRKDIRKNFWGIYTLQLIATILSLVLYTSLCLFFPGMQNMVAYILGLSLISKGMDISWLFQGLEDFRRITARNTTVKVLGVISIFLFVKTPGDLYLYVFLLTFFELLGQLSMWLPARPYIGKPQFDLSYAKKRLKPVILLFLPQVAISLYVTLDRTMLGALSSTNDVGIYDQALKIINILLTLVTSLGSVMLPRVSGLLSNGDHKAVNKMHELSFLIYNLVIFPIIAGLLIVNKDFVSFFLGKDFQEAYLAIAIMVFRMFFIGWTNIMGIQILIPHNKHREFMLSTTIPAVVSVGLNLLLIPPFGFVGASIVSVLTEALVWFIQLYFCLPYLKEVPILESLAKIVCASTMMYGLLLSAKPFLHFPPTLNVLVYAVIGGLIYLLAILVLKVVDVKELKQIIGEN

SEQ ID NO: 33 - protein, 332 aa, Streptococcus macedonicus
  misc_feature (49)..(49): Xaa can be any naturally occurring amino acid
MKKARNINLSLILIIGCIGVVLLHTTMPGFLETGRWNYSSYLYYLGTYXITLFFMVNGYLLLGKSKITYPYILHKIKWFLITVSSWTVIIWFLKRDFTINPIKKILASLIQKGYFFQFWFFGSLILIYLCLPILKKYLHSKRSYLYFLYVLTIIGLIFELINFLLQMPVQIYVIQTFRLWTXFFYYILGGFVAQFIIENLKSIFLGWMKIVSILLLLISPIILFFIAKTTYHNLFAEYFYDNLLVKVISLGLFLTLLTLTIDASKHRMIYLLSVQTMGVFIIHTYVMQIWQKLIGFNIVGAHLFFPVFTLVISFLISMILMKIPYINRIVKL

SEQ ID NO: 34 - protein, 366 aa, Streptococcus macedonicus
MYDYLIVGAGLSGAIFAQEATKRGKKVKVIDKRDHIGGNIYCEDVEGINVHKYGAHIFHTSNKKVWDYVNQFAEFNNYINSPIANYKGSLYNLPFNMNTFYAMWGTKTPQEVKDKIAEQTADMKDVEPKNLEEQAIKLIGPDIYEKLIKGYTEKQWGRSATDLPPFIIKRLPVRLTFDNNYFNDRYQGIPIGGYNVIIENMLGDVEVELGVDFFANREELEASAEKVVFTGMIDQYFDYKHGELEYRSLRFEHEVLDEENHQGNAVVNYTEREIPYTRIIEHKHFEYGTQPKTVITREYPADWKRGDEPYYPINDEKNNAMFAKYQEEAEKNDKVIFCGRLADYKYYDMHVVIERALEVVEKEFTI

SEQ ID NO: 35 - protein, 224 aa, Streptococcus macedonicus
MIEKNAPWVNNVYLITNGQKPDWLNLEHPKLKLVTHREFMPKEYLPTYNSAAIELNLHHIEGLSENYLYFNDDTYLIRDSQPSDFYKNGQPKLLAVYDALVPWPPFTNTYHNNVELIYRHFPNKKALKSSPWKFFNFRYGSLVLKNLLLLPWGPTRYVNQHLPVPMKKSTLAHLWEIEGETLDKTSRNPIRDYGVDVNQYICQHWQIESNQFYPMSKSFGETIG

SEQ ID NO: 36 - protein, 391 aa, Streptococcus macedonicus
MTQFTTELLNFLAQKQDIDEFFRTSLETAMNDLLQAELSAFLGYEPYDKLGYNSGNSRNGSYARKFETKYGTVQLSIPRDRNGNFSPALLPAYGRRDDHLEEMVIKLYQTGVTTREISDIIERMYGHHYSPATISNISKATQENVATFHERSLEANYSVLFLDGTYLPLRRGTVSKECIHIALGITPEGQKAVLGYEIAPNENNASWSTLLDKLQNQGIQQVSLVVTDGFKGLEEIINQAYPLAKQQRCLIHISRNLASKVKRADRAVILEQFKTIYRAENLEMAVQALENFISEWKPKYRKVMESLENTDNLLTFYQFPYQIWHSIYSTNLIESLNKEIKRQTKKKILFPNEEALGRYLVTLFEDYNFKQSQRTHKGFGQCADTLESLFD

SEQ ID NO: 37 - DNA, 300 bp, Streptococcus macedonicus
atgggaattgaaatttttattcgtaacccaaaaggcattaccttgactaaggatggcgttaggtttctttcttatgcgcgccaaattttagaacaaacagctcttttagaggaacgttataagagcaaaaatacaaaccgagaactgtttagcgtatcttcacagcactatgctttcgttgtcaatgcttttgtttcgcttttagaaggaacagatatgtcacgttatgagcttttccttcgcgaaacacgaacatatgaaattattgatgatgttaagaatttccgttcagaaattggc